(19) United States
(12) Patent Application Publication
(10) Pub. No.: US 2026/0341513 A1
(43) Pub. Date: Oct. 29, 2026

(54) METHODS AND SYSTEMS OF FACILITATING A HEALTH ASSESSMENT

(71) Applicant: Neoptio Health Incorporated, Vancouver (CA)

(72) Inventor: Christopher David McNamara, Nelson (CA)

(73) Assignee: Neoptio Health Incorporated, Vancouver (CA)

(21) Appl. No.: 19/693,631

(22) Filed: Jun. 1, 2026

Related U.S. Application Data
(63) Continuation of application No. PCT/IB2024/062256, filed on Dec. 5, 2024.
(60) Provisional application No. 63/606,569, filed on Dec. 5, 2023.

(51) Int. Cl. G16H 50/20; G16H 80/00

(52) U.S. Cl. CPC G16H 50/20; G16H 80/00

(57) ABSTRACT

The present disclosure provides a method of facilitating a health assessment. Further, the method may include receiving a medical inquiry. Further, the medical inquiry may be generated by a user. Further, the method may include analyzing the medical inquiry. Further, the method may include generating an assessment query based on the analyzing. Further, the generation of the assessment query may be based on one or more of a template and a first machine learning model. Further, the template includes a standardized assessment query. Further, the method may include transmitting the assessment query. Further, the method may include receiving a response from the healthcare communication infrastructure. Further, the response corresponds. Further, the method may include analyzing the response. Further, the method may include determining a diagnosis data based on the analyzing. Further, the method may include transmitting the diagnosis data.

### RELATED APPLICATIONS

This application is a US bypass continuation application of the PCT application PCT/IB2024/062256, tiled “METHODS AND SYSTEMS FOR AI-DRIVEN REFINEMENT OF MENTAL HEALTH DIAGNOSTICS AND PERSONALIZED TREATMENT”, filed on Dec. 5, 2024, which in turn claims benefit of the United States provisional patent application No. 63/606,569, titled “METHODS AND SYSTEMS FOR FACILITATING AUTOMATED HEALTH ASSESSMENT INITIATED THROUGH HEALTHCARE COMMUNICATION CHANNELS”, filed on Dec. 5, 2023, the entirety of which has been incorporated herein by reference.

### FIELD OF DISCLOSURE

The present invention relates generally to the field of data processing. More specifically, the present invention is methods and systems for facilitating a health assessment.

### BACKGROUND

The field of data processing is technologically important to several industries, business organizations, and/or individuals. In particular, the use of data processing is prevalent for methods and systems for facilitating automated health assessment initiated through healthcare communication channels.

The modern healthcare landscape is progressively being reshaped by digital technologies. Telehealth, telemedicine, and digital healthcare platforms have emerged as promising solutions to complement traditional healthcare delivery systems, aiming to provide more accessible, efficient, and cost-effective care. These technological advancements strive to bridge geographical and logistical barriers, ensuring individuals receive timely medical attention and support.

However, substantial challenges persist, particularly in the initial stages of healthcare communication and assessment across various medical disciplines, including mental health. Traditionally, individuals in need of healthcare support initiate contact through call centers, clinics, or direct lines to healthcare providers. These interactions often lack the depth of assessment necessary to provide accurate and timely insights into an individual's health status. The challenges are further compounded by long wait times, limited availability of healthcare providers, and potential inaccuracies in self-reported medical histories.

Moreover, collateral information, which includes data from other healthcare providers, medical records, and information from family members or other third parties, is often crucial for a comprehensive assessment of an individual's health. The existing systems frequently operate in silos, making the integration and utilization of collateral information a cumbersome process. This fragmentation hinders the seamless flow of critical health information among individuals, healthcare providers, and other stakeholders, thereby diminishing the efficacy of healthcare delivery.

Furthermore, the stigma associated with mental health often deters individuals from seeking timely help. When they do reach out, the interaction may not always capture the necessary breadth and depth of information required for accurate assessment and appropriate referral. This is especially problematic in the absence of a system that can aggregate and analyze collateral information alongside self-reported data to provide a more holistic view of an individual's mental health status.

Additionally, existing telehealth solutions tend to focus primarily on either physical health or mental health, with limited cross-disciplinary integration. There exists a dire need for an integrated system capable of seamlessly assessing and managing both mental and physical health concerns, providing a comprehensive view of an individual's health status.

Existing systems may not provide a comprehensive assessment as they might only focus on either physical or mental health but not both. They may lack a holistic approach that encompasses various medical disciplines.

Many current systems operate in silos and have limited capabilities in integrating and utilizing collateral information from other healthcare providers, medical records, and third parties, which is crucial for a thorough healthcare assessment. The assessment methods employed might be static and not adaptable to the unique needs and conditions of different individuals. They might not utilize dynamic questioning or machine learning to enhance the assessment process based on real-time responses from individuals. Existing systems might not offer a variety of interaction channels such as voice, web-based, and text-based interfaces to cater to different user preferences and needs. They might not provide personalized treatment recommendations or facilitate secure communications between individuals and healthcare providers for follow-up consultations and treatment planning. The fragmentation between different systems, such as Electronic Health Record (EHR) systems and telehealth platforms, might hinder the seamless flow of critical health information among individuals, healthcare providers, and other stakeholders.

Existing systems might not offer multilingual support, which is crucial in breaking down language barriers and ensuring that individuals, regardless of their linguistic background, can access and benefit from healthcare services. Some platforms might not operate in compliance with healthcare regulations and standards, which is crucial to ensure data privacy and security. Existing solutions might not facilitate community interactions among individuals with similar health conditions, which can promote peer support, shared learning, and better health management. They might not provide educational resources and support materials to individuals to promote health awareness and self-management.

Therefore, there is a need for improved methods and systems for facilitating automated health assessment initiated through healthcare communication channels that may overcome one or more of the above-mentioned problems and/or limitations.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method of facilitating a health assessment. Further, the method may include receiving, using a communication device, a medical inquiry from a healthcare communication infrastructure associated with a healthcare provider. Further, the medical inquiry may be generated by a user. Further, the method may include analyzing, using a processing device, the medical inquiry. Further, the method may include generating, using the processing device, an assessment query based on the analyzing. Further, the generation of the assessment query may be based on one or more of a template and a first machine learning model. Further, the template includes a standardized assessment query. Further, the method may include transmitting, using the communication device, the assessment query to the healthcare communication infrastructure. Further, the method may include receiving, using the communication device, a response from the healthcare communication infrastructure. Further, the response corresponds to the assessment query. Further, the method may include analyzing, using the processing device, the response. Further, the method may include determining, using the processing device, a diagnosis data based on the analyzing. Further, the method may include transmitting, using the processing device, the diagnosis data to the healthcare communication infrastructure.

The present disclosure provides a system for facilitating a health assessment. Further, the system may include a communication device. Further, the communication device may be configured to receive a medical inquiry from a healthcare communication infrastructure associated with a healthcare provider. Further, the medical inquiry may be generated by a user. Further, the communication device may be configured to transmit an assessment query to the healthcare communication infrastructure. Further, the communication device may be configured to receive a response from the healthcare communication infrastructure. Further, the response corresponds to the assessment query. Further, the communication device may be configured to transmit a diagnosis data to the healthcare communication infrastructure. Further, the system may include a processing device communicatively coupled with the communication device. Further, the processing device may be configured to analyze the medical inquiry. Further, the processing device may be configured to generate the assessment query based on the analyzing. Further, the generation of the assessment query may be based on one or more of a template and a first machine learning model. Further, the template includes a standardized assessment query. Further, the processing device may be configured to analyze the response. Further, the processing device may be configured to determine the diagnosis data based on the analyzing.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3A illustrates a flowchart of a method 300 of facilitating a health assessment, in accordance with some embodiments.

FIG. 3B illustrates a continuation of the flowchart of the method 300 of facilitating a health assessment, in accordance with some embodiments.

FIG. 4 illustrates a flowchart of a method 400 of facilitating a health assessment including receiving, using the communication device 902, an external data from the external data source device, in accordance with some embodiments.

FIG. 5 illustrates a flowchart of a method 500 of facilitating a health assessment including receiving, using the communication device 902, a treatment data from the healthcare provider device, in accordance with some embodiments.

FIG. 6 illustrates a flowchart of a method 600 of facilitating a health assessment including retrieving, using a storage device 1002, a relevant healthcare provider data from a plurality of healthcare provider data, in accordance with some embodiments.

FIG. 7 illustrates a flowchart of a method 700 of facilitating a health assessment including analyzing, using the processing device 904, the plurality of assessment queries, in accordance with some embodiments.

FIG. 8 illustrates a flowchart of a method 800 of facilitating a health assessment including generating, using the processing device 904, an appointment data indicative of a scheduled appointment of the user with the healthcare provider, in accordance with some embodiments.

FIG. 9 illustrates a block diagram of a system 900 of facilitating a health assessment, in accordance with some embodiments.

FIG. 10 illustrates a block diagram of the system 900 of facilitating a health assessment, in accordance with some embodiments.

FIG. 11 is a block diagram of a system 1100 for facilitating automated health assessment initiated through healthcare communication channels 1106, in accordance with some embodiments.

FIG. 12 illustrates the automated health assessment module 1104, in accordance with some embodiments.

FIG. 13 is a block diagram of a system 1300 for facilitating automated health assessment initiated through healthcare communication channels, in accordance with some embodiments.

FIG. 14 illustrates a flow diagram of the automated health assessment module 1304, in accordance with some embodiments.

FIG. 15 illustrates a block diagram of an automated health assessment module 1500, in accordance with some embodiments.

FIG. 16 illustrates an integration of the system 1600 for facilitating automated health assessment initiated through healthcare communication channels with external systems, in accordance with some embodiments.

FIG. 17 is a flowchart of a method 1700 for facilitating automated health assessment initiated through healthcare communication channels, in accordance with some embodiments.

FIG. 18 is a block diagram of a system 1800 for facilitating automated health assessment initiated through healthcare communication channels, in accordance with some embodiments.

FIG. 19 is a flowchart of a method 1900 for facilitating automated health assessment initiated through healthcare communication channels, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role-based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer, etc.) and/or possession of a machine readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human-readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data there between corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

According to some embodiments, a method for facilitating automated health assessment initiated through healthcare communication channels is disclosed. Further, the method may include a step of receiving, using a communication device, at least one medical assessment initiation request from at least one healthcare seeker device associated with at least one healthcare seeker. Further, the method may include a step of analyzing, using a processing device, the at least one medical assessment initiation request. Further, the method may include a step of determining, using the processing device, at least one health condition category of the at least one healthcare seeker based on the analyzing. Further, the method may include a step of generating, using the processing device, a plurality of personalized health assessment queries for the at least one healthcare seeker based on the at least one health condition category. Further, the method may include a step of transmitting, using the communication device, the plurality of personalized health assessment queries to the at least one healthcare seeker device. Further, the method may include a step of receiving, using the communication device, a plurality of assessment query responses of the at least one healthcare seeker from the at least one healthcare seeker device based on the plurality of personalized health assessment queries. Further, the method may include a step of analyzing, using the processing device, the plurality of assessment query responses. Further, the method may include a step of generating, using the processing device, at least one health report corresponding to the at least one health condition category based on the analyzing of the plurality of assessment query responses. Further, the method may include a step of transmitting, using the communication device, the at least one health report to the at least one healthcare seeker device. Further the method may include a step of storing, using a storage device, the at least one health report.

According to some aspects, a system for facilitating automated health assessment initiated through healthcare communication channels is disclosed. Accordingly, the system may include a processing device, a communication device, and a storage device. Further, the communication device may be communicatively coupled with the processing device. Further, the communication device may be communicatively coupled with the storage device. Further, the storage device may be communicatively coupled with the processing device. Further, the communication device may be configured for receiving at least one medical assessment initiation request from at least one healthcare seeker device associated with at least one healthcare seeker. Further, the communication device may be configured for transmitting a plurality of personalized health assessment queries to the at least one healthcare seeker device. Further, the communication device may be configured for receiving a plurality of assessment query responses of the at least one healthcare seeker from the at least one healthcare seeker device based on the plurality of personalized health assessment queries. Further, the communication device may be configured for transmitting at least one health report to the at least one healthcare seeker device. Further, the processing device may be configured for analyzing the at least one medical assessment initiation request. Further, the processing device may be configured for determining at least one health condition category of the at least one healthcare seeker based on the analyzing. Further, the processing device may be configured for generating the plurality of personalized health assessment queries for the at least one healthcare seeker based on the at least one health condition category. Further, the processing device may be configured for analyzing the plurality of assessment query responses. Further the processing device may be configured for generating the at least one health report corresponding to the at least one health condition category based on the analyzing of the plurality of assessment query responses. Further, the storage device may be configured for storing the at least one health report.

The present disclosure describes methods and systems for facilitating automated health assessment initiated through healthcare communication channels. Further, the disclosed system addresses the challenges in current healthcare communication and assessment practices across different medical disciplines, including mental health.

In accordance with one aspect of the system, a communication infrastructure is provided for receiving and managing communications from individuals seeking healthcare support. This infrastructure facilitates interactions through different healthcare communication channels including call center operations, clinic telecommunication systems, and healthcare provider direct lines.

In another aspect of the system, an automated health assessment module is operatively connected to the communication infrastructure. This module is configured to collect subjective medical history, and collateral information from other healthcare providers, medical records, and third parties, and track ongoing symptoms reported by individuals. The automated health assessment module comprises specialized sub-modules for different medical disciplines to ensure a holistic and personalized approach to healthcare assessment.

In a further aspect of the system, a communication channel is provided to facilitate interactions between individuals and the automated health assessment module. This communication channel comprises a voice channel for facilitating voice interactions, a web interface for facilitating online interactions, and a messaging interface for facilitating text-based interactions.

The mental health sub-module of the automated health assessment module is specifically configured to perform automated pre-diagnosis of mental health conditions based on the collected subjective medical history, collateral information, and ongoing symptoms. It is also configured to provide personalized mental health assessment and treatment recommendations and calculate medical and billing codes associated with identified mental health conditions.

The system is designed to provide a structured report of individuals' subjective medical history, collateral information, and ongoing symptoms to healthcare providers associated with the healthcare communication channels. It also facilitates secure communications between individuals and healthcare providers for follow-up consultations and treatment planning.

Furthermore, the system is configured to integrate with electronic health record systems to access and update medical records, incorporate collateral information, and facilitate appointment scheduling with healthcare providers based on assessment results and treatment recommendations.

By introducing a system that seamlessly integrates with existing healthcare communication channels and provides automated, comprehensive, and personalized health assessments, the present system strives to enhance the accuracy and efficiency of healthcare assessments and subsequent interventions across various medical disciplines.

The system aims to provide a comprehensive and integrated platform for collecting subjective medical history, tracking ongoing symptoms, and incorporating collateral information. By facilitating meaningful interactions between individuals and healthcare providers across a range of medical disciplines, the system strives to enhance the accuracy and efficiency of healthcare assessments and subsequent interventions.

The system for automated health assessment is initiated through various healthcare communication channels. This system is engineered to bridge significant gaps in current healthcare communication and assessment practices across different medical disciplines, including mental health.

The system comprises a communication infrastructure capable of receiving and managing communications from individuals seeking healthcare support. This infrastructure facilitates interactions through different healthcare communication channels including but not limited to call center operations, clinic telecommunication systems, and healthcare provider direct lines. It is equipped to handle voice, web-based, and text-based interactions, thus offering a versatile platform for individuals to initiate healthcare communication.

Operatively connected to the communication infrastructure is an automated health assessment module. This module is configured to employ a versatile combination of deterministic static pre-defined questions, which may include branching logic based on user responses, and dynamic questioning facilitated in real-time by language model algorithms, including large language models (LLMs). This approach enhances the depth, adaptability, and personalization of health assessments across various medical disciplines. The system is designed to operate using static, dynamic, or a hybrid questioning method, allowing for continuous evolution and improvement in line with advancements in language processing and machine learning technologies. This innovative approach allows for a more thorough and tailored assessment experience, catering to the unique needs and conditions of each individual. Future embodiments of this module may encompass a range of advanced methodologies to further revolutionize automated health assessments. These could include emotion recognition and sentiment analysis using Al for deeper mental health insights, interactive VR/AR-based assessments for more engaging and informative experiences, integration of biometric data for comprehensive health profiling, blockchain technology for enhanced data security, predictive analytics for proactive health management, and the incorporation of IOT device data for continuous and dynamic health monitoring. These advancements aim to elevate the depth, accuracy, and personalization of health assessments in a rapidly evolving digital health landscape.

The automated health assessment module houses specialized sub-modules for different medical disciplines to ensure a holistic and personalized approach to healthcare assessment. These include: a) Mental Health Sub-Module: Performs automated pre-diagnosis of mental health conditions based on collected subjective medical history, collateral information and ongoing symptoms. Utilizing machine learning algorithms, it provides personalized mental health assessment and treatment recommendations. It also facilitates secure communication between said individuals and mental health professionals through end-to-end encryption, ensuring the privacy and security of sensitive health information; b) Neurology Sub-Module: For assessing and tracking neurological conditions; c) Psychiatry Sub-Module: For diagnosing, treating, and preventing mental emotional, and behavioral disorders; d) Behavioral Medicine Sub-Module: For addressing the interaction of behavioral, psychosocial, and biomedical factors; e) Sleep Medicine Sub-Module: For assessing and managing sleep disorder; f) Addiction Medicine Sub-Module: For managing substance abuse and addiction; g) Geriatric Medicine Sub-Module: For addressing mental health challenges in the elderly population; h) Pediatric Psychiatry or Psychology Sub-Module: For addressing mental health in children and adolescents; i) Psychotherapy Sub-Module: For facilitating psychotherapeutic interventions; j) Family Medicine or General Practice Sub-Module: For providing initial mental health assessments and referrals; k) Occupational Therapy Sub-Module: For assessing the intersection between daily activities, physical health, and mental health; l) Endocrinology Sub-Module: For evaluating the impact of hormonal imbalances on mental health; m) Additional Sub-Modules: Configured to address other medical disciplines not explicitly listed, ensuring a comprehensive approach to healthcare assessment.

A communication channel is provided to facilitate interactions between individuals and the automated health assessment module. This communication channel comprises: a) Voice Channel: Facilitates voice interactions between individuals and the automated health assessment module, enabling individuals to verbally communicate their symptoms, medical history, and other relevant information; b) Web Interface: Facilitates online interactions, enabling individuals to input their medical history, symptoms, and other relevant information through a user-friendly web interface; c) Messaging Interface: Facilitates text-based interactions, allowing individuals to communicate their medical history, symptoms, and other relevant information through text messaging.

The system is also configured to facilitate community interactions among individuals with similar health conditions. By promoting peer support, shared learning, and enhanced health management, individuals can engage with a supportive community that aids in better understanding and managing their conditions.

The system is designed to provide a structured report(s) of individuals' subjective medical history, collateral information, and ongoing symptoms to healthcare providers associated with the healthcare communication channels. It facilitates secure communications between individuals and healthcare providers for follow-up consultations and treatment planning. The structured report can be shared electronically with healthcare providers, ensuring a seamless flow of critical health information for accurate assessment and intervention.

The system is configured to integrate with electronic health record systems to access and update medical records, incorporate collateral information, and facilitate appointment scheduling with healthcare providers based on assessment results and treatment recommendations.

The system operates in compliance with healthcare regulations and standards to ensure data privacy and security. By employing robust encryption protocols and adhering to healthcare data privacy regulations, the system ensures that all communications and data exchanges within the system are secure and private.

The communication channel within the system is configured to provide multilingual support to cater to a diverse user base, thereby enhancing accessibility and inclusivity in healthcare communication. This feature is crucial in breaking down language barriers and ensuring that individuals, regardless of their linguistic background, can access and benefit from the automated health assessment system.

By introducing a system that seamlessly integrates with existing healthcare communication channels and provides automated, comprehensive, and personalized health assessments, the present system strives to enhance the accuracy and efficiency of healthcare assessments and subsequent interventions across various medical disciplines.

The system for automated health assessment initiated through healthcare communication channels, comprising: a) a communication infrastructure for receiving and managing communications from individuals; b) an automated health assessment module, operatively connected to said communication infrastructure, configured to collect subjective medical history, and collateral information, and track ongoing symptoms; c) a communication channel for facilitating interactions between said individuals and said automated health assessment module.

Further, the healthcare communication channels comprise: a) call center operations; b) a clinic telecommunication systems; c) a healthcare provider direct lines.

Further, the communication channel comprises: a) a voice channel for facilitating voice interactions; b) a web interface for facilitating online interactions; c) a messaging interface for facilitating text-based interactions.

The automated health assessment module is configured to employ a versatile combination of deterministic static pre-defined questions, which may include branching logic based on user responses, and dynamic questioning facilitated in real-time by language model algorithms, including large language models (LLMs). This approach enhances the depth, adaptability, and personalization of health assessments across various medical disciplines. The system is designed to operate using static, dynamic, or a hybrid questioning method, allowing for continuous evolution and improvement in line with advancements in language processing and machine learning technologies.

The automated health assessment module is configured to collect subjective medical history, collateral information from other healthcare providers, medical records and third parties, and track ongoing symptoms from said individuals across various medical disciplines.

The automated health assessment module comprises specialized sub-modules for different medical disciplines.

The specialized sub-modules of the automated health assessment module are configured to address distinct medical disciplines, comprising at least one of the following sub-modules: a) a mental health sub-module for performing assessments and tracking conditions related to mental health; b) a neurology sub-module for assessing and tracking neurological conditions; c) a psychiatry sub-module for diagnosing, treating, and preventing mental, emotional, and behavioral disorders; d) a behavioral medicine sub-module for addressing the interaction of behavioral, psychosocial, and biomedical factors; e) a sleep medicine sub-module for assessing and managing sleep disorders; f) an addiction medicine sub-module for managing substance abuse and addiction; g) a geriatric medicine sub-module for addressing mental health challenges in the elderly population; h) a pediatric psychiatry or psychology sub-module for addressing mental health in children and adolescents; i) a psychotherapy sub-module for facilitating psychotherapeutic interventions; j) a family medicine or general practice sub-module for providing initial mental health assessments and referrals; k) an occupational therapy sub-module for assessing the intersection between daily activities, physical health, and mental health; l) an endocrinology sub-module for evaluating the impact of hormonal imbalances on mental health; m) additional sub-modules configured to address other medical disciplines not explicitly listed.

The mental health sub-module is configured to perform automated pre-diagnosis of mental health conditions based on collected subjective medical history, collateral information, and ongoing symptoms; provide personalized mental health additional assessment and treatment recommendations utilizing machine learning algorithms facilitate secure communication between said individuals and mental health professionals through end-to-end encryption; track and analyze mental health progress over time using machine learning algorithms and predictive analytics; provide resources and support for mental health education and awareness; calculate medical and billing codes associated with mental health services.

The automated health assessment module is configured to provide a structured report of said individuals' subjective medical history, collateral information, and ongoing symptoms to healthcare providers associated with said healthcare communication channels.

The automated health assessment module is configured to dynamically adjust the assessment process based on real-time responses from said individuals, allowing for a tailored assessment experience.

The automated health assessment module is configured to provide immediate feedback to said individuals based on the collected subjective medical history and ongoing symptoms, enhancing user engagement and understanding.

The automated health assessment module is configured to generate a unique user profile for each individual to facilitate personalized assessment and treatment recommendations.

The automated health assessment module is configured to facilitate secure communications between said individuals and healthcare providers for follow-up consultations and treatment planning, employing encryption and compliance with healthcare data privacy regulations.

The automated health assessment module is configured to integrate with electronic health record systems to access and update medical records and incorporate collateral information of said individuals enhancing the continuity of care and the accuracy of the health assessment.

The automated health assessment module is configured to facilitate appointment scheduling with healthcare providers based on the assessment results and treatment recommendations.

The automated health assessment module is configured to provide educational resources and support materials to said individuals to promote health awareness and self-management.

The automated health assessment module is configured to facilitate community interactions among individuals with similar health conditions to promote peer support, shared learning, and enhanced health.

The communication infrastructure is configured to manage communications across multiple channels including voice, messaging, and online interfaces, to facilitate accessibility and user engagement.

The communication channel is configured to provide multilingual support to cater to a diverse user base, enhancing accessibility and inclusivity in healthcare communication.

The automated health assessment module is configured to operate in compliance with healthcare regulations and standards to ensure data privacy and security.

The system aims to address several prominent issues in the healthcare sector, particularly around the initial stages of healthcare communication and assessment across various medical disciplines, including mental health. Traditional methods of initiating healthcare support, such as through call centers, clinics, or direct lines to healthcare providers, often lack the depth of assessment necessary to provide accurate and timely insights into an individual's health status.

This is further exacerbated by long wait times, limited availability of healthcare providers, and potential inaccuracies in self-reported medical histories. Moreover, the fragmentation in the current systems makes the integration and utilization of collateral information (such as data from other healthcare providers, medical records, and information from family or other third parties) a cumbersome process, hindering the seamless flow of critical health information among individuals, healthcare providers, and other stakeholders. This fragmentation diminishes the efficacy of healthcare delivery. Additionally, the stigma associated with mental health often deters individuals from seeking timely help, and when they do reach out, the interaction may not always capture the necessary breadth and depth of information required for accurate assessment and appropriate referral. Existing telehealth solutions tend to focus primarily on either physical health or mental health, with limited cross-disciplinary integration, which creates a dire need for an integrated system capable of seamlessly assessing and managing both mental and physical health concerns to provide a comprehensive view of an individual's health status. The system for automated health assessment is initiated via various healthcare communication channels, targeting existing challenges in healthcare communication and assessment across diverse medical disciplines, including mental health. The system embodies a communication infrastructure, facilitating interactions through different channels like call centers, clinic telecommunication systems, and healthcare provider direct lines. Connected to this infrastructure is an Automated Health Assessment Module, configured to collect subjective medical history, and collateral information, and track ongoing symptoms reported by individuals. This module is adept at employing deterministic static pre-defined questions, which may include branching logic based on user responses, and dynamic questioning facilitated in real-time by language model algorithms, including large language models (LLMs). This approach enhances the depth, adaptability, and personalization of health assessments across various medical disciplines. The system is designed to operate using static, dynamic, or a hybrid questioning method, allowing for continuous evolution and improvement in line with advancements in language processing and machine learning technologies. It hosts specialized sub-modules for different medical disciplines, each tailored to collect pertinent data and furnish personalized health assessment and treatment recommendations. Furthermore, the system promotes structured reporting and secure communications between individuals and healthcare providers, and seamlessly integrates with electronic health record systems for a smooth transition of critical health information. By fostering accurate and efficient healthcare assessments and subsequent interventions across various medical disciplines, this invention strives to bridge the gaps in current healthcare communication and assessment practices.

Comprehensive Assessment across Disciplines: The system houses specialized sub-modules for different medical disciplines, ensuring a holistic approach to healthcare assessment. It covers both physical and mental health, addressing a broader spectrum of healthcare needs compared to some existing solutions.

Integration of Collateral Information: It is designed to collect and integrate subjective medical history, and collateral information from other healthcare provider's medical records, and third parties. This thorough collection and integration of data enable a more complete understanding of an individual's health status.

Dynamic Assessment Methods: The system employs a versatile combination of deterministic static pre-defined questions, which may include branching logic based on user responses, and dynamic questioning facilitated in real-time by language model algorithms, including large language models (LLMs). This approach enhances the depth, adaptability, and personalization of health assessments across various medical disciplines. The system is designed to operate using static, dynamic, or a hybrid questioning method, allowing for continuous evolution and improvement in line with advancements in language processing and machine learning technologies.

Versatile Interaction Channels: It offers multiple interaction channels, including voice, web-based, and text-based interactions, facilitating a broader range of user preferences and needs, thereby enhancing user engagement and accessibility.

Personalized Treatment Recommendations: The specialized sub-modules, such as the mental health sub-module, are configured to provide personalized assessment and treatment recommendations, fostering a more tailored approach to individual healthcare needs.

Seamless System Integration: The system is configured to integrate seamlessly with electronic health record systems to access and update medical records, facilitate appointment scheduling, and ensure a seamless flow of critical health information among all stakeholders.

Multilingual Support: The communication channel within the system provides multilingual support, catering to a diverse user base and breaking down language barriers that might exist in other systems

Regulatory Compliance for Data Privacy and Security: It operates in compliance with healthcare regulations and standards, employing robust encryption protocols to ensure data privacy and security, which is critical in the healthcare domain.

Community Interaction Feature: The system facilitates community interactions among individuals with similar health conditions, promoting peer support, shared learning, and better health management which might not be a feature in many existing solutions.

Educational Resources and Support: The system provides educational resources and support materials to promote health awareness and self-management, adding a layer of support that might be missing in other platforms.

Structured Reporting and Secure Communications: It provides structured reporting to healthcare providers and facilitates secure communications for follow-up consultations and treatment planning, ensuring a continuous care pathway.

Through these features and functionalities, the system aims to provide a more comprehensive, accessible, and personalized healthcare assessment experience, striving to enhance the accuracy and efficiency of healthcare assessments and subsequent interventions across various medical disciplines. By addressing the gaps in current healthcare communication and assessment practices, this system could potentially offer a more robust and user-friendly solution compared to existing platforms.

Communication Infrastructure: Receives and manages communications from individuals. Includes call center operations, clinic telecommunication systems, and healthcare provider direct lines.

Automated Health Assessment Module: Collects subjective medical history, and collateral information, and tracks ongoing symptoms. Employs a versatile combination of deterministic static pre-defined questions, which may include branching logic based on user responses, and dynamic questioning facilitated in real-time by language model algorithms, including large language models (LLMs). This approach enhances the depth, adaptability, and personalization of health assessments across various medical disciplines. The system is designed to operate using static, dynamic, or a hybrid questioning method, allowing for continuous evolution and improvement in line with advancements in language processing and machine learning technologies.

Specialized Sub-Modules include: a) Mental Health Sub-Module; b) Neurology Sub-Module; c) Psychiatry Sub-Module; d) Behavioral Medicine Sub-Module; e) Sleep Medicine Sub-Module; f) Addiction Medicine Sub-Module; g) Geriatric Medicine Sub-Module; h) Pediatric Psychiatry or Psychology Sub-Module; i) Psychotherapy Sub-Module; j) Family Medicine or General Practice Sub-Module; k) Occupational Therapy Sub-Module; l) Endocrinology Sub-Module; m) Additional Sub-Modules for other medical disciplines not explicitly listed.

Communication Channel include: a) Voice Channel; b) Web Interface; c) Messaging Interface.

Community Interaction Feature: facilitates community interactions among individuals with similar health conditions.

Structured Reporting and Secure Communications: a) Provides structured reports to healthcare providers; b) Facilitates secure communications for follow-up consultations and treatment planning.

Integration with Electronic Health Record Systems: a) Accesses and updates medical records; b) Incorporates collateral information and facilitates appointment scheduling.

Security and Compliance Features: a) Ensures compliance with healthcare regulations and standards; b) Provides robust encryption protocols for data privacy and security.

Multilingual Support: Provides multilingual support to cater to a diverse user base

Educational Resources and Support Materials: Provides resources for health awareness and self-management.

Each of these components (elements) plays a crucial role in ensuring that the system effectively addresses the problems identified in healthcare communication and assessment practices, offering a more comprehensive, personalized, and user-friendly solution.

The system aims to enhance healthcare assessments and communications.

Initiation of Communication: Individuals initiate communication through various channels managed by the Communication Infrastructure. This could be through call center operations, clinic telecommunication systems, or direct lines to healthcare providers.

Channeling Communication: The Communication Channel processes these communications, categorizing them based on their medium: voice, online, or text-based interactions.

Automated Health Assessment: The Automated Health Assessment Module receives the communications channeled through the communication infrastructure. This module collects subjective medical history, and collateral information, and tracks ongoing symptoms from individuals. The module employs a versatile combination of deterministic static pre-defined questions, which may include branching logic based on user responses, and dynamic questioning facilitated in real-time by language model algorithms, including large language models (LLMs). This approach enhances the depth, adaptability, and personalization of health assessments across various medical disciplines. The system is designed to operate using static, dynamic, or a hybrid questioning method allowing for continuous evolution and improvement in line with advancements in language processing and machine learning technologies.

Specialized Assessments: Within the Automated Health Assessment Module, Specialized Sub-Modules come into play based on the medical discipline relevant to the individual's needs. For example, mental health concerns would be directed to the Mental Health Sub-Module, while neurological issues would be directed to the Neurology Sub-Module. Each sub-module conducts a more focused and tailored assessment.

Community Interaction: The Community Interaction Feature may be leveraged to connect individuals with similar health conditions, promoting peer support, shared learning, and enhanced health management.

Structured Reporting: Post-assessment, the system creates structured reports that encapsulate the collected data and assessment results. These reports are channeled back to healthcare providers via the Structured Reporting and Secure Communications feature.

Integration and Appointment Scheduling: The system's Integration with Electronic Health Record Systems feature facilitates the updating of medical records and scheduling of follow-up appointments based on assessment results and treatment recommendations.

Security and Compliance: Throughout this process, the Security and Compliance Features ensure that all communications and data exchanges are securely encrypted and compliant with healthcare regulations and standards.

Multilingual and Educational Support: Multilingual Support ensures that individuals can interact with the system in a language they are comfortable with. Additionally, Educational Resources and Support Materials are provided to individuals to promote health awareness and self-management.

Continuous Improvement: The interaction and feedback from the various components, along with the real-time responses from individuals, can be utilized for continuous improvement of the system, enhancing its adaptability and effectiveness over time.

The orchestration of these components provides a holistic, automated, and personalized approach to healthcare assessment and communication, aiming to significantly enhance the accuracy and efficiency of healthcare assessments and subsequent interventions across various medical disciplines.

The components and elements of the system are meticulously designed to work both individually and together to create a seamless and efficient system for automated health assessments through various healthcare communication channels. Here's how they function:

Communication Infrastructure: a) Individually: It acts as the initial point of contact, receiving and managing communications from individuals seeking healthcare support; b) Collectively: It channels these communications to the Automated Health Assessment Module for further action, serving as a bridge between individuals and the health assessment system.

Communication Channel a) Individually: It categorizes the communications based on their medium voice, online, or text-based, and facilitates the interaction between individuals and the Automated Health Assessment Module; b) Collectively: It works with the Communication Infrastructure to ensure that communications are correctly channeled and with the Automated Health Assessment Module to ensure that assessments are conducted smoothly.

Automated Health Assessment Module: a) Individually: It collects essential health information, conducts assessments, and generates personalized feedback based on the data received; b) Collectively: It interacts with specialized sub-modules for a more focused assessment, and with other components for reporting, secure communication, and integration with electronic health records.

Specialized Sub-Modules: a) Individually: Each sub-module conducts a tailored assessment based on its specialized medical discipline; b) Collectively: They contribute to a holistic assessment by covering a wide range of medical disciplines, ensuring a comprehensive health assessment.

Community Interaction Feature: a) Individually: It facilitates peer support and shared learning among individuals with similar health conditions; b) Collectively: It enhances the user experience by providing a supportive community, which could be beneficial for mental and emotional well-being.

Structured Reporting and Secure Communications: a) Individually: It generates structured reports and ensures secure communication between individuals and healthcare providers; b) Collectively: It interacts with the Automated Health Assessment Module to receive assessment data and with Electronic Health Record Systems for integration and updates.

Integration with Electronic Health Record Systems: a) Individually: It updates medical records and facilitates appointment scheduling; b) Collectively: It works with the Structured Reporting and Secure Communications feature to ensure seamless sharing and updating of health information.

Security and Compliance: a) Individually: It ensures data privacy, security, and compliance with healthcare regulations and standards; b) Collectively: It provides a secure environment for all interactions and data exchanges within the system.

Multilingual Support: a) Individually: It breaks down language barriers, enabling a diverse user base to interact with the system; b) Collectively: It enhances the accessibility and inclusivity of the system.

Educational Resources and Support Materials: a) Individually: They provide essential information to promote health awareness and self-management; b) Collectively: They enrich the user experience by providing valuable resources for better health management.

The synergy among these components and elements enables the system to perform its desired function of providing automated, comprehensive, and personalized health assessments, which in turn, aims to enhance the accuracy and efficiency of healthcare assessments and subsequent interventions across various medical disciplines.

Through a well-orchestrated interaction among its components, the system addresses the notable gaps in current healthcare communication and assessment practices. Creating this system involves a multi-step process that encompasses system design, development, testing, and implementation. The step-by-step guide on how to make the Automated Health Assessment System includes: a) Research and Planning: identify the gaps in existing healthcare communication and assessment practices, Research the technical requirements and regulatory compliance necessary for developing a healthcare assessment system, and conduct a market analysis to understand the needs and preferences of the target user base; b) System Architecture Design: design the architecture of the communication infrastructure to handle various types of communications (voice, online, text-based), outline the structure of the Automated Health Assessment Module including the specialized sub-modules for different medical disciplines, and plan the integration with Electronic Health Record Systems and other external systems; c) Development of Communication Infrastructure: develop the communication infrastructure to receive and manage communications from individuals, ensure the infrastructure is capable of handling different types of communications efficiently, and implement security protocols to ensure data privacy and compliance with healthcare regulations; d) Development of Automated Health Assessment Module: develop the core automated health assessment module to collect essential health information, implement the functionality to employ a versatile combination of deterministic static pre-defined questions, which may include branching logic based on user responses, and dynamic questioning facilitated in real-time by language model algorithms, including large language models (LLMs), and develop specialized sub-modules for different medical disciplines based on the outlined structure; e) Development of Communication Channel: develop communication channels for voice interactions, online interactions, and text-based interactions, and ensure the channels are user-friendly and accessible to a diverse user base; f) Integration with External Systems: develop the functionality to integrate with Electronic Health Record Systems, and ensure seamless data exchange between the system and external systems; g) Development of Additional Features: develop the Community Interaction Feature to facilitate peer support and shared learning, and create Educational Resources and Support Materials to promote health awareness and self-management; h) Testing and Quality Assurance: conduct thorough testing of each component and the system as a whole to ensure it functions as intended, ensure compliance with healthcare regulations and standards, and address any bugs or issues identified during testing; i) User Interface and Experience Design: design a user-friendly interface for the web and messaging interfaces, and ensure the user interface is intuitive and provides a good user experience; j) Implementation and Deployment: deploy the system in a controlled environment for initial use and feedback, and collect feedback from users and healthcare providers to identify areas for improvement; k) Continuous Improvement and Updates: make necessary updates and improvements based on feedback and evolving regulatory requirements, and ensure the system stays updated with the latest technology advancements and healthcare practices; l) Monitoring and Support: provide ongoing support for system maintenance and troubleshooting. Monitor system performance and user engagement to ensure it continues to meet the needs of its users and achieves its intended purpose.

By following these steps meticulously, adhering to healthcare regulations, and engaging with healthcare professionals for insights, you can develop, implement, and continuously improve the Automated Health Assessment System as outlined in the system.

This system is designed to offer a comprehensive and personalized healthcare assessment experience through various communication channels. A patient in a remote rural area with limited access to healthcare may use the system's web interface to enter the medical history and current symptoms. The system's automated health assessment module employs dynamic questioning to uncover nuanced details of their condition, such as the severity of symptoms related to a chronic illness. The system integrates with the local clinic's electronic health record system to schedule a telemedicine follow-up based on the urgency of the symptoms, bridging the gap caused by geographical barriers.

Further, an individual experiencing a mental health crisis may engage with the system via a messaging interface. The mental health sub-module conducts a pre-diagnosis based on the user input and guides the user through a series of questions to assess immediate risk. Simultaneously, it flags the interaction for urgent review by a mental health professional and schedules an emergency telehealth session while providing immediate resources and support materials.

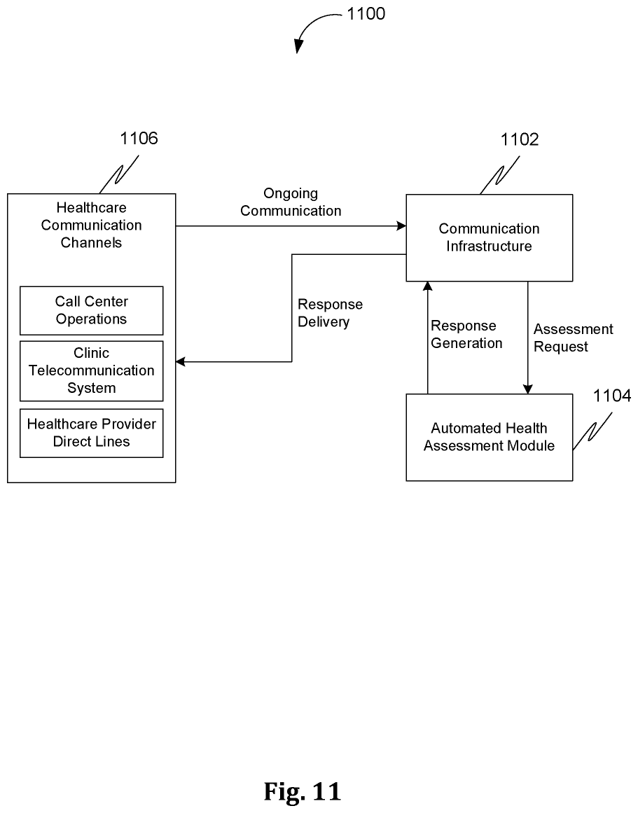

Referring now to figures, FIG. 11 is a block diagram of a system 1100 for facilitating automated health assessment initiated through healthcare communication channels 1106, in accordance with some embodiments. Accordingly, the system 1100 may include a communication infrastructure 1102 and an automated health assessment module 1104. Further, the communication infrastructure 1102 may be configured for receiving a plurality of healthcare support requests from a plurality of healthcare support seekers. Further, the communication infrastructure 1102 may be configured for managing communications and sending a plurality of responses corresponding to the plurality of healthcare support requests to the plurality of healthcare support seekers. Further, the communication infrastructure 1102 may be configured for communicating through a plurality of healthcare communication channels 1106. Further, the plurality of healthcare communication channels 1106 may include, but may not be limited to, call center operations, clinic telecommunication systems, and healthcare provider direct lines. Further, in some embodiments, the communication infrastructure 1102 may be equipped to handle voice, web-based, and text based interactions to offer a versatile platform for the plurality of healthcare support seekers to initiate healthcare communication. Further, the communication infrastructure 1102 may be communicatively coupled with the automated health assessment module 1104. Further the automated health assessment module 1104 may be configured to collect a subjective medical history of each healthcare support seeker of the plurality of healthcare support seekers, collateral information from a plurality of additional healthcare provider's medical records, and third parties, and track ongoing symptoms reported by the plurality of healthcare support seekers. Further, the automated health assessment module 1104 may be configured to generate a plurality of deterministic static pre-defined questions and a plurality of dynamic questions. Further, the automated health assessment module 1104 may include at least one language model algorithm (or large language model) for the generating of the plurality of dynamic questions. Further, the plurality of dynamic questions may be generated based on real-time responses from the plurality of healthcare support seekers.

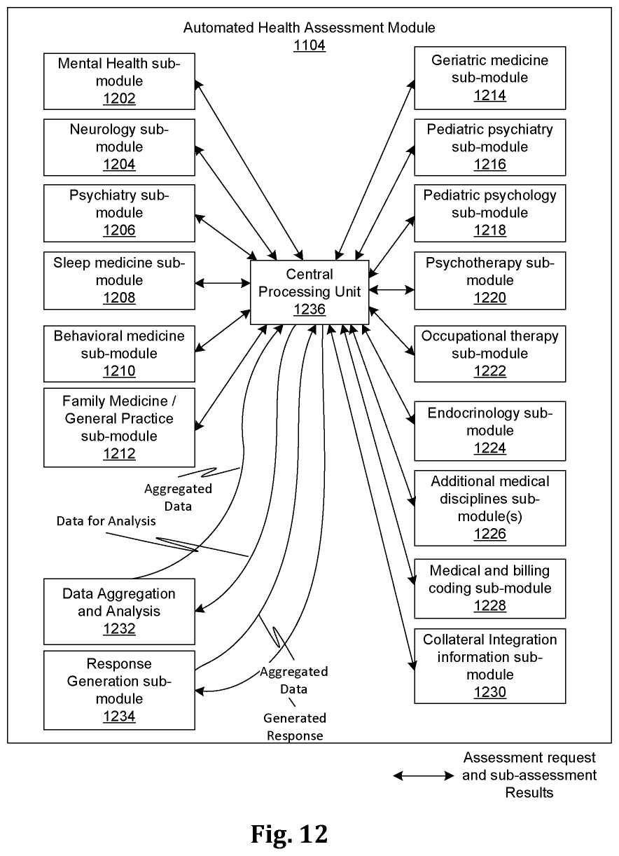

FIG. 12 illustrates the automated health assessment module 1104, in accordance with some embodiments.

Accordingly, the automated health assessment module 1104 may include a central processing unit 1236. Further, the automated health assessment module may include a mental health sub-module 1202 configured for performing automated pre-diagnosis of mental health conditions based on collecting a subjective medical history of a healthcare support seeker, collateral information, and ongoing symptoms. Further, the mental health submodule 1202 may utilize a plurality of machine learning algorithms to provide personalized mental health assessment and treatment recommendations. Further, the mental health submodule 1202 may secure communication between the plurality of healthcare support seekers and a plurality of mental health professionals through end-to-end encryption, ensuring privacy and security of sensitive health information. Further, the automated health assessment module 1104 may include a neurology sub-module 1204 configured for assessing and tracking neurological conditions. Further, the automated health assessment module 1104 may include a psychiatry sub-module 1206 configured for diagnosing, treating, and preventing mental, emotional, and behavioral disorders. Further, the automated health assessment module 1104 may include a behavioral medicine sub-module 1210 configured for addressing the interaction of behavioral, psychosocial, and biomedical factors. Further, the automated health assessment module 1104 may include a sleep medicine sub-module 1208 configured for assessing and managing sleep disorders. Further, the automated health assessment module 1104 may include an addiction medicine sub-module configured for managing substance abuse and addiction. Further, the automated health assessment module 1104 may include a geriatric medicine sub-module 1214 configured for addressing mental health challenges in the elderly population. Further, the automated health assessment module 1104 may include a pediatric psychiatry 1216 or psychology sub-module 1218 configured for addressing mental health in children and adolescents. Further, the automated health assessment module 1104 may include a psychotherapy sub-module 1220 configured for facilitating psychotherapeutic interventions. Further, the automated health assessment module 1104 may include a family medicine or general practice sub-module 1212 configured for providing initial mental health assessments and referrals. Further, the automated health assessment module 1104 may include an occupational therapy sub-module 1222 configured for assessing the intersection between daily activities, physical health, and mental health. Further, the automated health assessment module 1104 may include an endocrinology sub-module 1224 configured for evaluating the impact of hormonal imbalances on mental health. Further, the automated health assessment module 1104 may include additional sub-modules 1226 configured to address other medical disciplines not explicitly listed, ensuring a comprehensive approach to healthcare assessment. Further, the automated health assessment module 1104 may include a medical and billing coding sub-module 1228. Further, the automated health assessment module 1104 may include a collateral integration information sub-module 1230. Further, the automated health assessment module 1104 may include a data aggregation and analysis module 1232. Further, the automated health assessment module 1104 may include a response generation sub-module 1234 configured for generating the structured report.

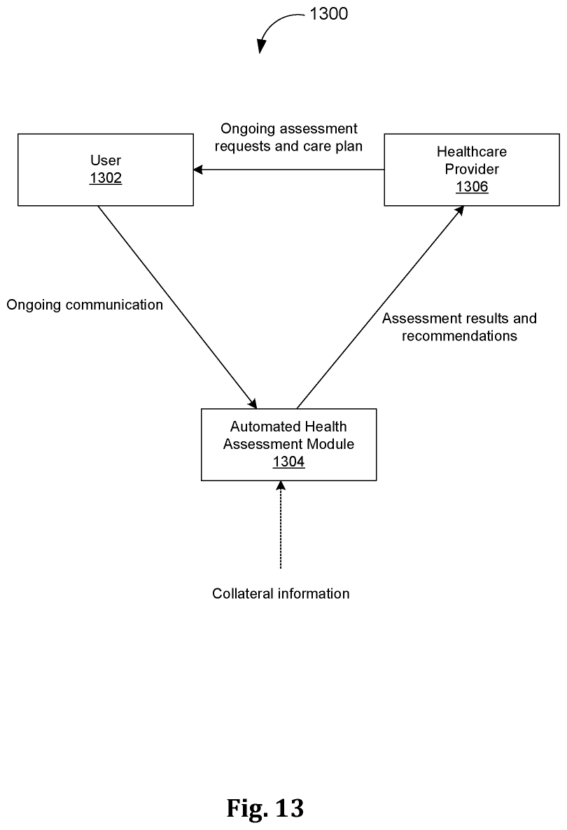

FIG. 13 is a block diagram of a system 1300 for facilitating automated health assessment initiated through healthcare communication channels, in accordance with some embodiments.

Accordingly, the system 1300 may be configured to provide a structured report of a subjective medical history, collateral information, and ongoing symptoms of a healthcare seeker (or user) 1302 generated by an automated health assessment module 1304 to a plurality of healthcare providers 1306 associated with the healthcare communication channels. Further, the system 1300 facilitates secure communications between the plurality of healthcare seekers 1302 and the plurality of healthcare providers 1306 for follow-up consultations and treatment planning. Further, the structured report may be shared electronically with the plurality of healthcare providers 1306, ensuring a seamless flow of critical health information for accurate assessment and intervention.

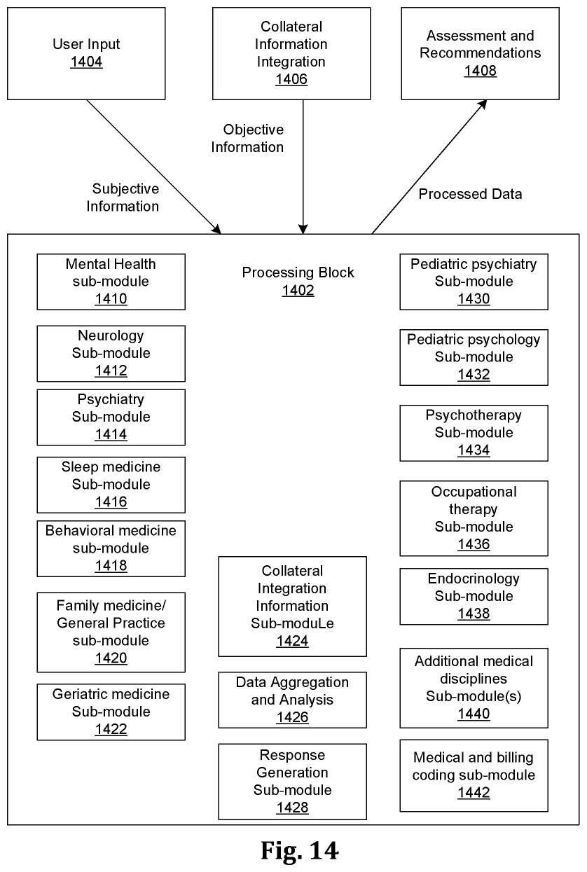

FIG. 14 illustrates a flow diagram of the automated health assessment module 1304, in accordance with some embodiments.

Accordingly, the automated health assessment module 1304 may include a processing block 1402 may include a plurality of sub-modules. Further, the plurality of sub-modules may include a mental health sub-module 1410, a neurology sub-module 1412, a psychiatry sub-module 1414, a sleep medicine sub-module 1416, a behavioral sub-module 1418, a family medicine/general practice sub-module 1420, a geriatric medicine sub-module 1422, a collateral integration information sub-module 1424, a data aggregation and analysis sub-module 1426, a response generation sub-module 1428, a pediatric psychiatry sub-module 1430, a pediatric psychology sub-module 1432, a psychotherapy sub-module 1434, an occupational therapy sub-module 1436, a endocrinology sub-module 1438, additional medical disciplines sub-module 1440, and a medical and billing coding sub-module 1442. Further, the processing block 1402 may be configured to receive a subject information from a user input device 1404. Further, the processing block 1402 may be configured to receive an objective information from a collateral information integration 1406. Further, the processing block 1402 may be configured to generate an assessment and recommendations 1408 based on the processed data.

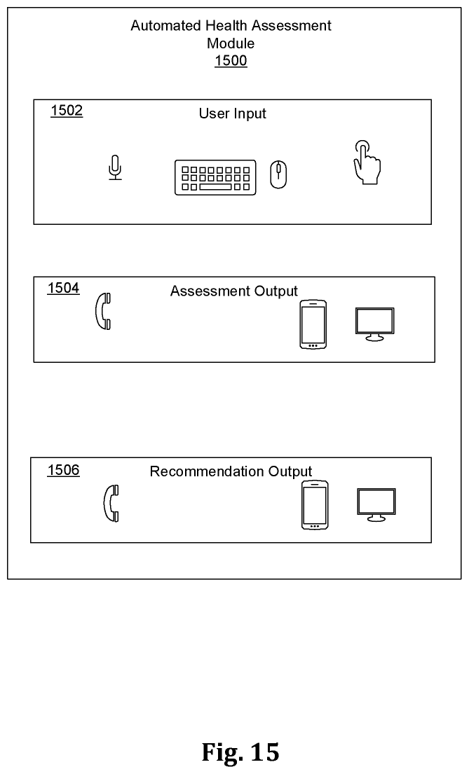

FIG. 15 illustrates a block diagram of an automated health assessment module 1500, in accordance with some embodiments.

Accordingly, the automated health assessment module 1500 may include a plurality of sub-modules. Further, the plurality of sub-modules may include a first module 1502, a second module 1504, and a third module 1506. Further, the first module 1502 may be configured to facilitated communication of a user input. Further, the second module 1504 may be configured to facilitate communication of an assessment output. Further, the third module 1506 may be configured to facilitate communication of a recommendation output.

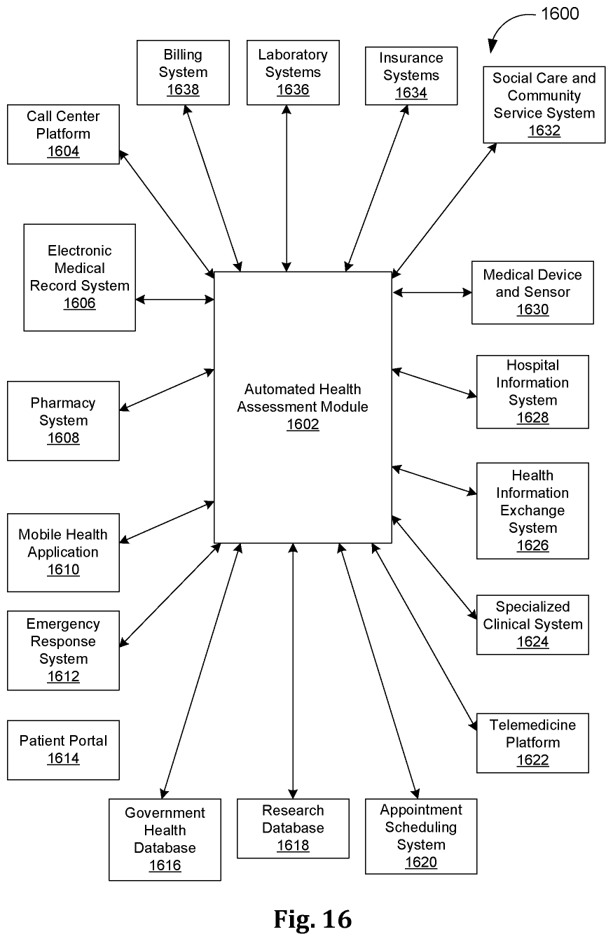

FIG. 16 illustrates an integration of the system 1600 for facilitating automated health assessment initiated through healthcare communication channels with external systems, in accordance with some embodiments.

Accordingly, the system 1600 comprises an automated health assessment module 1602. Further, the automated health assessment module 1602 may be configured to integrate with a plurality of external systems. Further, the plurality of external systems may include a call center platform 1604, an electronic medical record system 1606, a pharmacy system 1608, a mobile health application 1610, an emergency response system 1612, a patient portal 1614, a government health database 1616, a research database 1618, an appointment scheduling system 1620, a telemedicine platform 1622, a specialized clinical system 1624, an health information exchange system 1626, a hospital information system 1628, a medical device and sensor 1630, a social care and community service system 1632, an insurance system 1634, a laboratory system 1636, a billing system 1638.

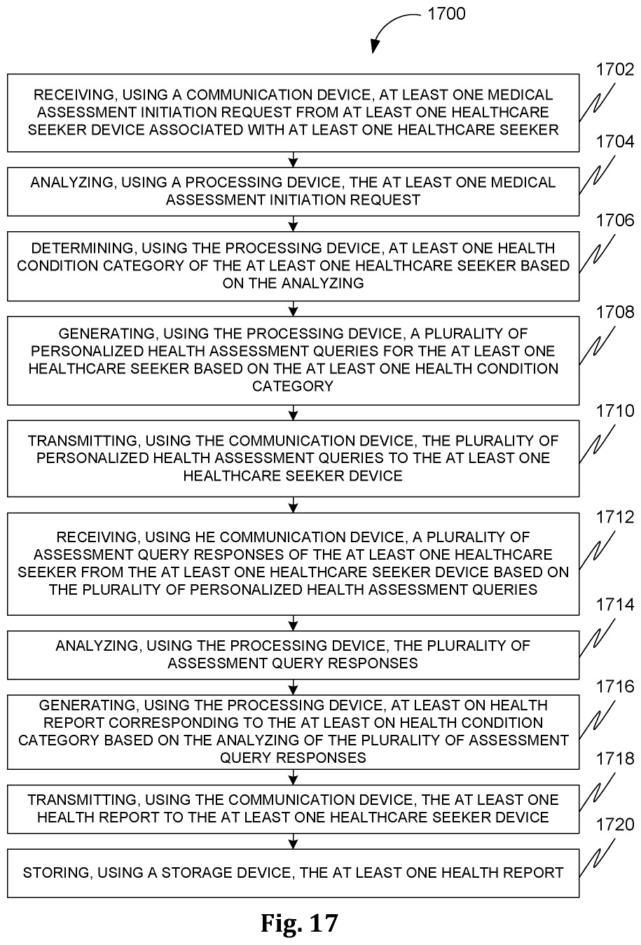

FIG. 17 is a flowchart of a method 1700 for facilitating automated health assessment initiated through healthcare communication channels, in accordance with some embodiments.

Further, the method 1700 may include a step 1702 of receiving, using a communication device 1802, at least one medical assessment initiation request from at least one healthcare seeker device associated with at least one healthcare seeker. Further the at least one healthcare seeker device may include, but may not be limited to, a smartphone, a laptop, a desktop, a tablet computer, etc. Further, the at least one medical assessment initiation request may indicate that the at least one healthcare seeker may want to initiate a healthcare assessment of the at least one healthcare seeker.

Further, the method 1700 may include a step 1704 of analyzing, using a processing device 1804, the at least one medical assessment initiation request.

Further, the method 1700 may include a step 1706 of determining, using the processing device 1804, at least one health condition category of the at least one healthcare seeker based on the analyzing.

Further, the method 1700 may include a step 1708 of generating, using the processing device 1804, a plurality of personalized health assessment queries for the at least one healthcare seeker based on the at least one health condition category.

Further, the method 1700 may include a step 1710 of transmitting, using the communication device 1802, the plurality of personalized health assessment queries to the at least one healthcare seeker device.

Further, the method 1700 may include a step 1712 of receiving, using the communication device 1802, a plurality of assessment query responses of the at least one healthcare seeker from the at least one healthcare seeker device based on the plurality of personalized health assessment queries.

Further, the method 1700 may include a step 1714 of analyzing, using the processing device 1804, the plurality of assessment query responses.

Further the method 1700 may include a step 1716 of generating, using the processing device 1804, at least one health report corresponding to the at least one health condition category based on the analyzing of the plurality of assessment query responses.

Further, the method 1700 may include a step 1718 of transmitting, using the communication device 1802, the at least one health report to the at least one healthcare seeker device.

Further, the method 1700 may include a step 1720 of storing, using a processing device 1806, the at least one health report.

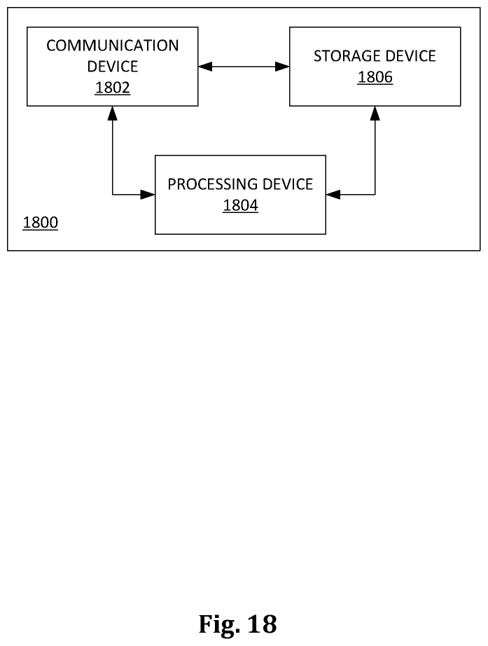

FIG. 18 is a block diagram of a system 1800 for facilitating automated health assessment initiated through healthcare communication channels, in accordance with some embodiments.

Accordingly, the system 1800 may include a processing device 1804, a communication device 1802, and a storage device 1806. Further, the communication device 1802 may be communicatively coupled with the processing device 1804. Further, the communication device 1802 may be communicatively coupled with the storage device 1806. Further, the storage device 1806 may be communicatively coupled with the processing device 1804.

Further, the communication device 1802 may be configured for receiving at least one medical assessment initiation request from at least one healthcare seeker device associated with at least one healthcare seeker. Further, the at least one healthcare seeker device may include, but may not be limited to, a smartphone, a laptop, a desktop, a tablet computer, etc. Further, the at least one medical assessment initiation request may indicate that the at least one healthcare seeker may want to initiate a healthcare assessment of the at least one healthcare seeker. Further, the communication device 1802 may be configured for transmitting a plurality of personalized health assessment queries to the at least one healthcare seeker device. Further, the communication device 1802 may be configured for receiving a plurality of assessment query responses of the at least one healthcare seeker from the at least one healthcare seeker device based on the plurality of personalized health assessment queries. Further, the communication device 1802 may be configured for transmitting at least one health report to the at least one healthcare seeker device. Further, the processing device 1804 may be configured for analyzing the at least one medical assessment initiation request. Further, the processing device 1804 may be configured for determining at least one health condition category of the at least one healthcare seeker based on the analyzing. Further, the processing device 1804 may be configured for generating the plurality of personalized health assessment queries for the at least one healthcare seeker based on the at least one health condition category. Further, the processing device 1804 may be configured for analyzing the plurality of assessment query responses. Further, the processing device 1804 may be configured for generating the at least one health report corresponding to the at least one health condition category based on the analyzing of the plurality of assessment query responses.

Further, the storage device 1806 may be configured for storing the at least one health report.

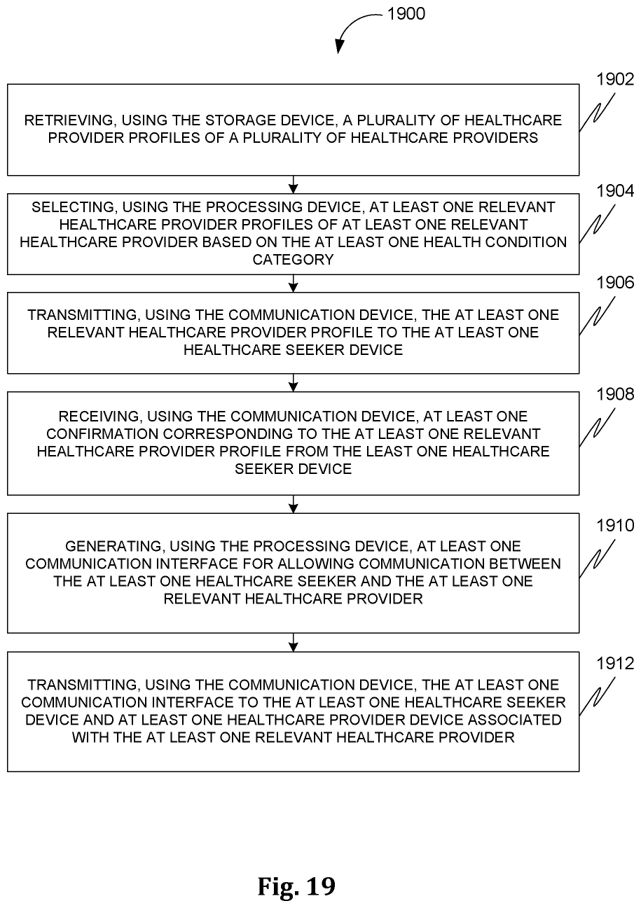

FIG. 19 is a flowchart of a method 1900 for facilitating automated health assessment initiated through healthcare communication channels, in accordance with some embodiments.

Further, the method 1900 may include a step 1902 of retrieving, using the storage device 1806, a plurality of healthcare provider profiles of a plurality of healthcare providers.

Further, the method 1900 may include a step 1904 of selecting, using the processing device 1804, at least one relevant healthcare provider profile of at least one relevant healthcare provider based on the at least one health condition category

Further, the method 1900 may include a step 1906 of transmitting, using the communication device 1802, the at least one relevant healthcare provider profile to the at least one healthcare seeker device.

Further, the method 1900 may include a step 1908 of receiving, using the communication device 1802, at least one confirmation corresponding to the at least one relevant healthcare provider profile from the at least one healthcare seeker device.

Further, the method 1900 may include a step 1910 of generating, using the processing device 1804, at least one communication interface for allowing communication between the at least one healthcare seeker and the at least one relevant healthcare provider.

Further, the method 1900 may include a step 1912 of transmitting, using the communication device 1802, the at least one communication interface to the at least one healthcare seeker device and at least one healthcare provider device associated with the at least one relevant healthcare provider. Further, the at least one healthcare provider device may include, but may not be limited to, a smartphone, a laptop, a desktop, a tablet computer, etc.

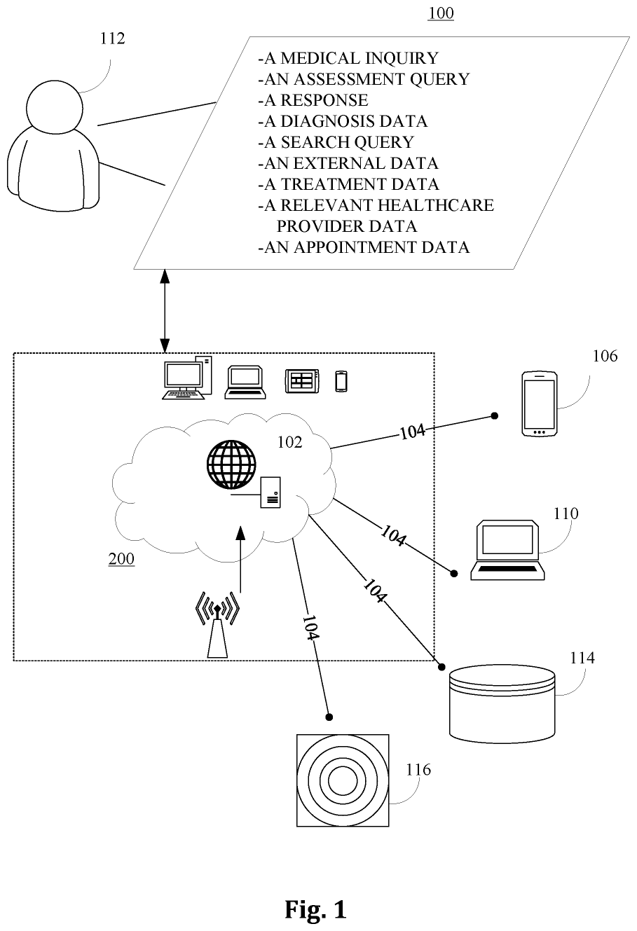

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to the Internet. Further, users of the online platform 100 may include relevant parties such as, but not limited to, end-users, administrators, service providers, and service consumers, and so on. Accordingly, in some instances, electronic devices operated by the one or more relevant parties may be in communication with the platform.

A user 112, such as the one or more relevant parties, may access online platform 100 through a web-based software application or browser. The web-based software application may be embodied as, for example, but not be limited to, a website, a web application, a desktop application, and a mobile application compatible with a computing device 200.

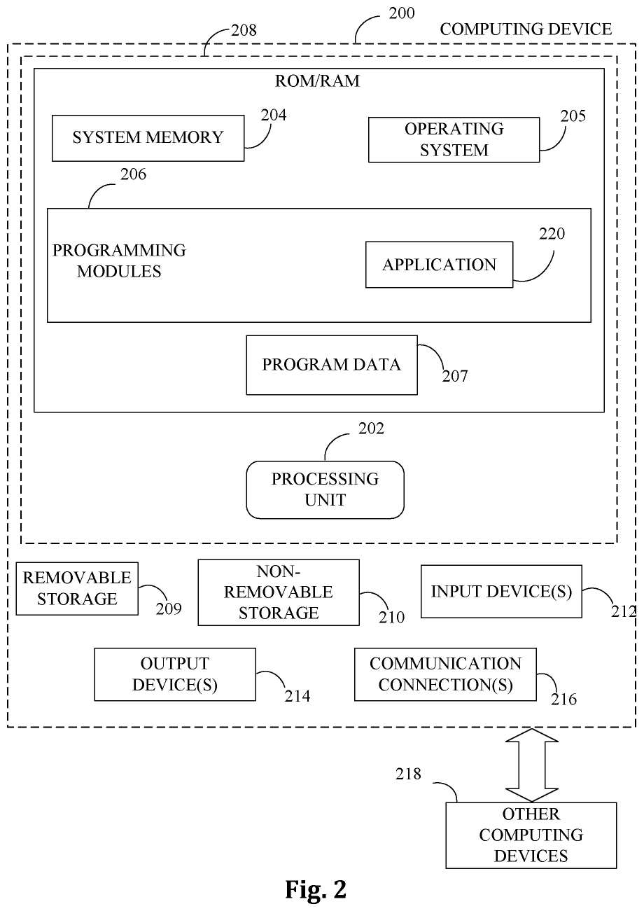

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable, and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include but is not limited to, RAM, ROM, electrically erasable read-only memory (EEPROM), flash memory or other memory technology, CD-ROM, digital versatile disks (DVD), or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example, but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

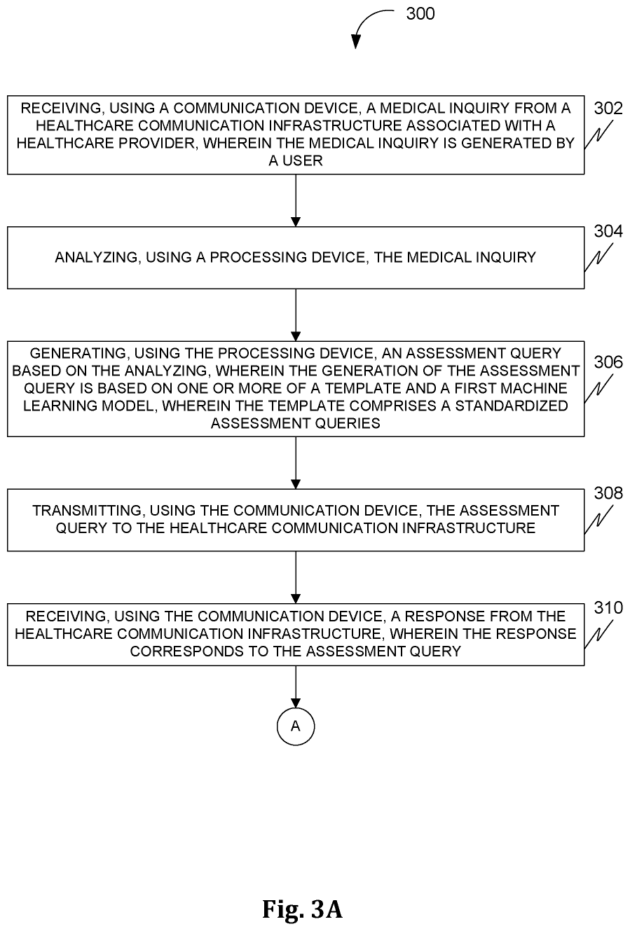

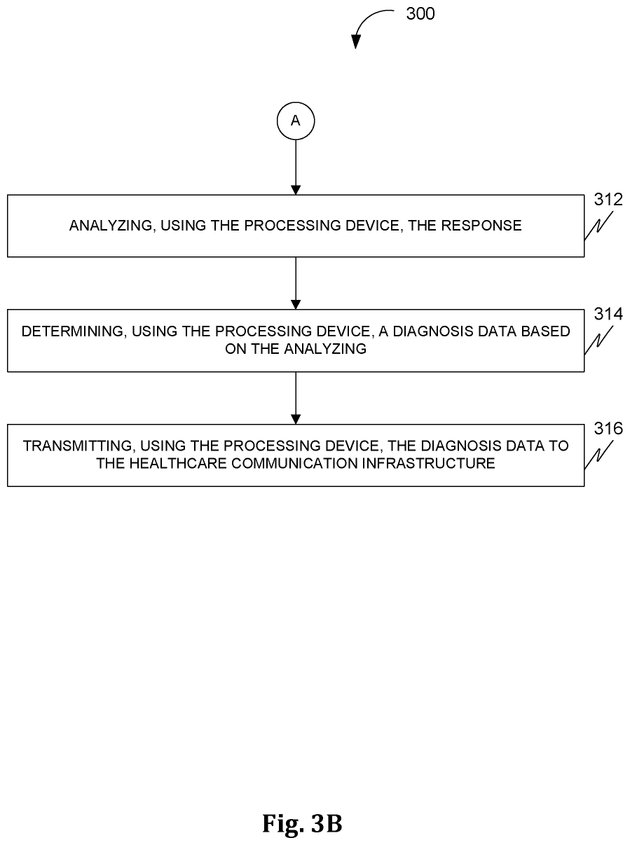

FIG. 3A AND FIG. 3B illustrate a flowchart of a method 300 of facilitating a health assessment, in accordance with some embodiments.

In some embodiments, the health assessment may be facilitated by an automated health assessment module. In some embodiments, the health assessment includes an assessment of one or more a mental health condition and a physical health condition.

Accordingly, the method 300 may include a step 302 of receiving, using a communication device 902, a medical inquiry from a healthcare communication infrastructure 906 associated with a healthcare provider. Further, the medical inquiry may be generated by a user.

In some embodiments, the communication device 902 may be an electronic device which may be configured to one or more of receive or transmit a data. In some embodiments, one or more of the receiving and transmitting of the data may be wireless.

In some embodiments, the medical inquiry includes one or more of a personal data associated with the user, an indication of a need for medical attention, and a medical malady.

In some embodiments, the healthcare communication infrastructure 906 facilitates communication between the healthcare provider and the user.

In some embodiments, the healthcare communication infrastructure 906 includes one or more of a healthcare provider direct lines, a call center operation, and a clinic telecommunication system. In some embodiments, the healthcare communication infrastructure 906 includes one or more of a voice channel infrastructure 906, a web interface infrastructure 906, and a messaging infrastructure 906.

In some embodiments, the healthcare provider includes one or more of a hospital, a clinic, and medical assistance provider.

In some embodiments, receiving of the medical inquiry may be based on communication of the user with the healthcare provider using the healthcare communication infrastructure 906.

Further, the method 300 may include a step 304 of analyzing, using a processing device 904, the medical inquiry.

In some embodiments, the processing device 904 may be an electronic device which may be configured to execute a set of instructions.

In some embodiments, the analysis of the medical inquiry may be based on a machine learning model.

Further, the method 300 may include a step 306 of generating, using the processing device 904, an assessment query based on the analyzing. Further, the generation of the assessment query may be based on one or more of a template and a first machine learning model.

In some embodiments, the assessment query includes of a personalized query for the user based on the analyzing of the medical inquiry.

In some embodiments, the first machine learning model includes one or more of a supervised learning model, an unsupervised learning model, a semi-supervised learning model, and a reinforcement learning model. In some embodiments, the first machine learning model includes a generative machine learning model. In some embodiments, the large language model includes a Generative Pre-trained model (GPT model).

In some embodiments, the first machine learning model may be trained on training data corresponding to a functionality. Further, the training data includes an input training data and an output training data. Further, the output training data may be generated by an implementation of the functionality based on the input training data.

Further, the template includes a standardized assessment query. Further, the method 300 may include a step 308 of transmitting, using the communication device 902, the assessment query to the healthcare communication infrastructure 906.

In some embodiments, the assessment query may be generated in real time.

In some embodiments, the assessment query includes of a query which may be configured to determine a state of one or more of a physical health and a mental health of the user.

In some embodiments, the receiving and transmitting of the one or more of the medical inquiry, the assessment query, and the response may be facilitated by an Interactive Voice Response.

Further, the method 300 may include a step 310 of receiving, using the communication device 902, a response from the healthcare communication infrastructure 906. Further, the response corresponds to the assessment query.

In some embodiments, the receiving of the may be facilitated by an emotion recognition system. Further, the generation of the diagnosis data may be further based on a sentiment analysis based on a third machine learning model.

In some embodiments, the assessment query may be presented to the user using one or more of a virtual reality and an artificial reality apparatus.

Further, the method 300 may include a step 312 of analyzing, using the processing device 904, the response. Further, the method 300 may include a step 314 of determining, using the processing device 904, a diagnosis data based on the analyzing.

In some embodiments, the diagnosis data includes a structured report of the user based on one or more of the responses, a medical history, and a collateral information. In some embodiments, generation of the diagnosis data may be further based on a medical history, a collateral information, and an ongoing symptom. In some embodiments, the diagnosis data further includes a medical billing code associated with a diagnosed medical malady.

Further, the method 300 may include a step 316 of transmitting, using the processing device 904, the diagnosis data to the healthcare communication infrastructure 906.

In some embodiments, the method 300 may further include determining, using the processing device 904, a healthcare domain associated with the medical inquiry based on the analyzing.

In some embodiments, the healthcare domain corresponds to an assessment module. In some embodiments, the assessment module includes one or more of a mental health module, a neurology module, a psychiatry module, a sleep medicine module, a behavioral module, a family medicine and general practice module, a geriatric module, a pediatric psychiatry, a pediatric psychology, a psychotherapy module, an occupational therapy module, an endocrinology module, and an additional medicine discipline module.

Further, the assessment query includes two or more assessment queries associated with the healthcare domain. Further, the response includes two or more responses. Further, each of the two or more responses corresponds to each of the two or more assessment questions.

In some embodiments, the analyzing of the responses may be further based on a second machine learning. Further, the second machine learning model may be configured to receive an input based on the response. Further, the second machine learning model may be configured to generate the diagnosis data as an output.

In some embodiments, the first machine learning model includes a large language model. Further, the medical inquiry includes a symptom of a medical condition of the user. Further, the large language model may be configured to receive the symptom as an input. Further, the large language model may be configured to generate the assessment query as output based on the input.

In some embodiments, the analyzing of the medical inquiry may be based on a speech recognition model.

In some embodiments, the speech recognition model includes a natural language processing unit.

Further, the speech recognition model may be configured to determine one or more of a medical malady or a symptom of medical malady. Further, the generation of the assessment query may be based on one or more of the medical malady and the symptom of medical malady.

Further, in some embodiments, the method 300 may include generating, using the processing device 904, a user interface which may be configured to facilitate communication between the user and the healthcare provider. Further, in some embodiments, the method 300 may include transmitting, using the communication device 902, the communication interface to each of the healthcare communication infrastructure 906, and the relevant healthcare provider.

In some embodiments, the method 300 may further include receiving, using the communication device 902, a medical sensor data generated by a medical sensor associated with the user. Further, the generation of the diagnosis data may be further based on the medical sensor data.

In some embodiments, the communication between the communication device 902 and the healthcare communication infrastructure 906 may be encrypted.

In some embodiments, the method 300 may further include integrating, using the processing device 904, an external electronic health record systems for accessing and updating one or more of a medical records, a collateral information, and an appointment scheduling.

Further, in some embodiments, the method 300 may include receiving, using the communication device 902, a biometric data associated with the user. Further, in some embodiments, the method 300 may include generating, using the processing device 904, a user profile corresponding to the user. Further, the user profile includes the biometric data and the diagnosis data. Further, in some embodiments, the method 300 may include transmitting, using the communication device 902, the user profile to one or more of the healthcare communication infrastructure 906 and the healthcare provider device.

In some embodiments, the method 300 may further include transmitting, using the communication device 902, an educational data to the healthcare communication infrastructure 906. Further, the education data includes a resource for mental health education and awareness.

In some embodiments, the method 300 may further include receiving, using the communication device 902, a feedback data from the healthcare communication infrastructure 906. Further, the feedback data may be indicative of an improvement associated with a user experience.

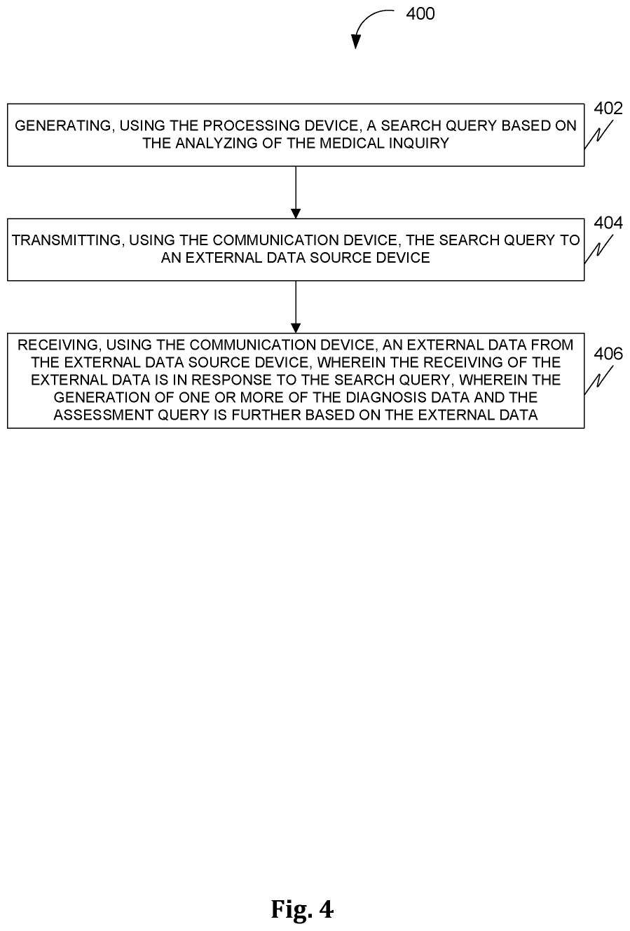

FIG. 4 illustrates a flowchart of a method 400 of facilitating a health assessment including receiving, using the communication device 902, an external data from the external data source device, in accordance with some embodiments.

Further, in some embodiments, the method 400 further may include a step 402 of generating, using the processing device 904, a search query based on the analyzing of the medical inquiry. Further, in some embodiments, the method 400 further may include a step 404 of transmitting, using the communication device 902, the search query to an external data source device. Further, in some embodiments, the method 400 further may include a step 406 of receiving, using the communication device 902, an external data from the external data source device.

In some embodiments, the external data includes one or more of a collateral information, a medical history, and a medical record. In some embodiments, the collateral information includes a data from the healthcare providers, a medical information from a medical report, and a medical information from a third party.

Further, the receiving of the external data may be in response to the search query. Further, the generation of one or more of the diagnosis data and the assessment query may be further based on the external data.

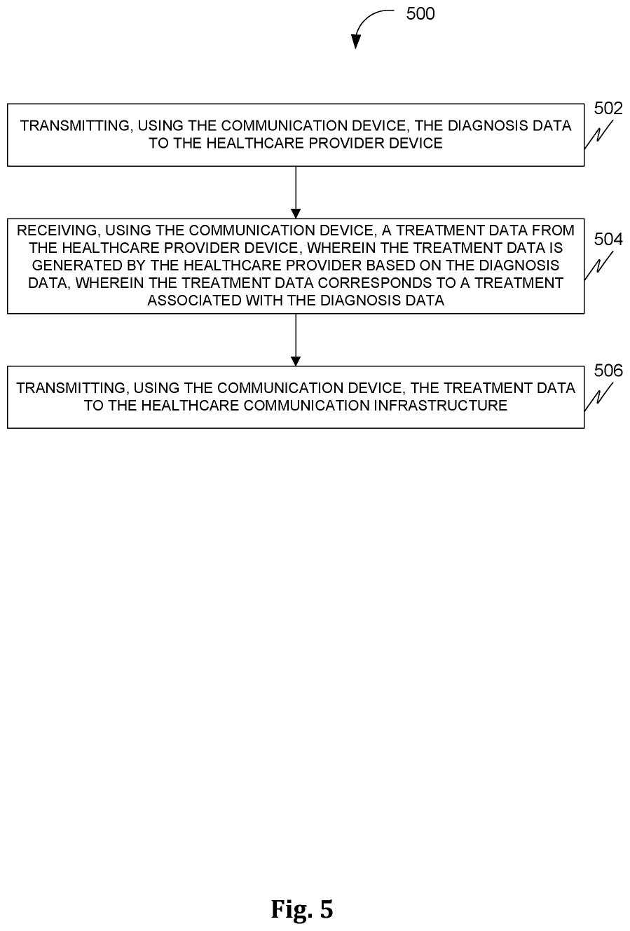

FIG. 5 illustrates a flowchart of a method 500 of facilitating a health assessment including receiving, using the communication device 902, a treatment data from the healthcare provider device, in accordance with some embodiments.

Further, in some embodiments, the method 500 further may include a step 502 of transmitting, using the communication device 902, the diagnosis data to the healthcare provider device. Further, in some embodiments, the method 500 may include a step 504 of receiving, using the communication device 902, a treatment data from the healthcare provider device. Further, the treatment data may be generated by the healthcare provider based on the diagnosis data. Further, the treatment data corresponds to a treatment associated with the diagnosis data. Further, in some embodiments, the method 500 may include a step 506 of transmitting, using the communication device 902, the treatment data to the healthcare communication infrastructure 906.

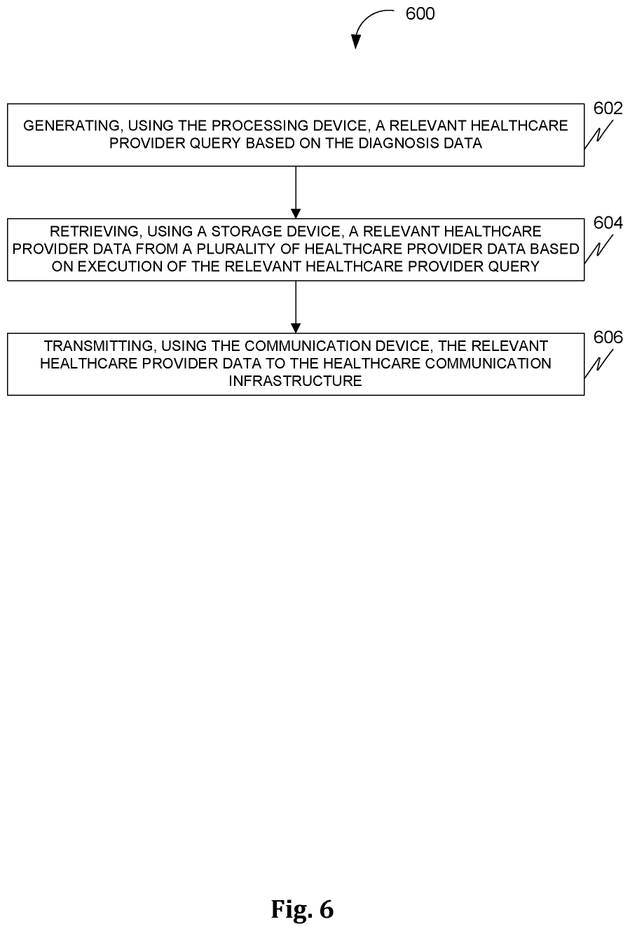

FIG. 6 illustrates a flowchart of a method 600 of facilitating a health assessment including retrieving, using a storage device 1002, a relevant healthcare provider data from a plurality of healthcare provider data, in accordance with some embodiments.

Further, in some embodiments, the method 600 further may include a step 602 of generating, using the processing device 904, a relevant healthcare provider query based on the diagnosis data. Further, in some embodiments, the method 600 further may include a step 604 of retrieving, using a storage device 1002, a relevant healthcare provider data from two or more healthcare provider data based on execution of the relevant healthcare provider query.

In some embodiments, the storage device 1002 includes a non-volatile memory.

Further, in some embodiments, the method 600 may include a step 606 of transmitting, using the communication device 902, the relevant healthcare provider data to the healthcare communication infrastructure 906.

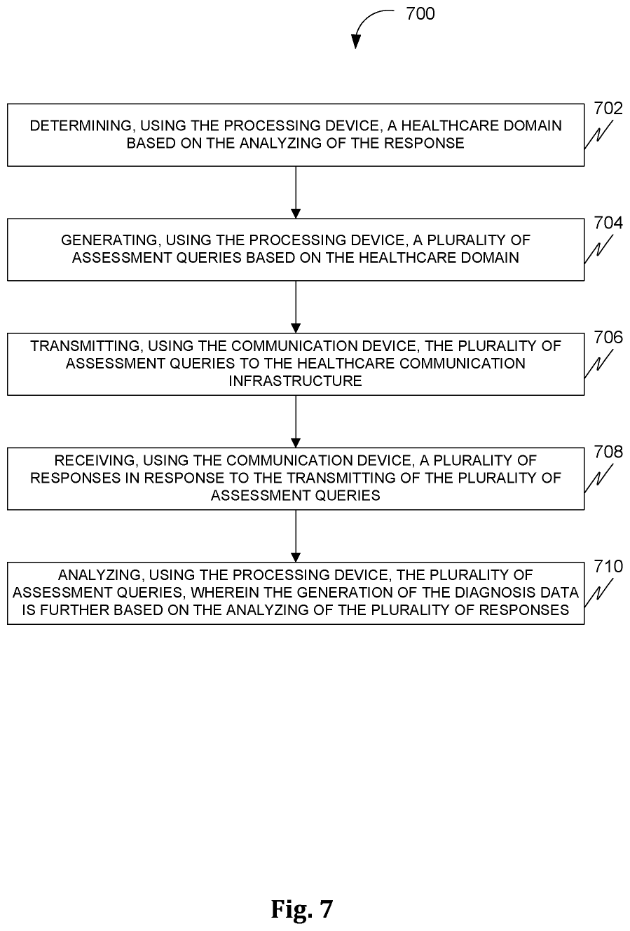

FIG. 7 illustrates a flowchart of a method 700 of facilitating a health assessment including analyzing, using the processing device 904, the plurality of assessment queries, in accordance with some embodiments.

Further, in some embodiments, the method 700 may include a step 702 of determining, using the processing device 904, a healthcare domain based on the analyzing of the response. Further, in some embodiments, the method 700 further may include a step 704 of generating, using the processing device 904, two or more assessment queries based on the healthcare domain. Further, in some embodiments, the method 700 further may include a step 706 of transmitting, using the communication device 902, the two or more assessment queries to the healthcare communication infrastructure 906. Further, in some embodiments, the method 700 further may include a step 708 of receiving, using the communication device 902, two or more responses in response to the transmitting of the two or more assessment queries. Further, in some embodiments, the method 700 further may include a step 710 of analyzing, using the processing device 904, the two or more assessment queries. Further, the generation of the diagnosis data may be further based on the analyzing of the two or more responses.

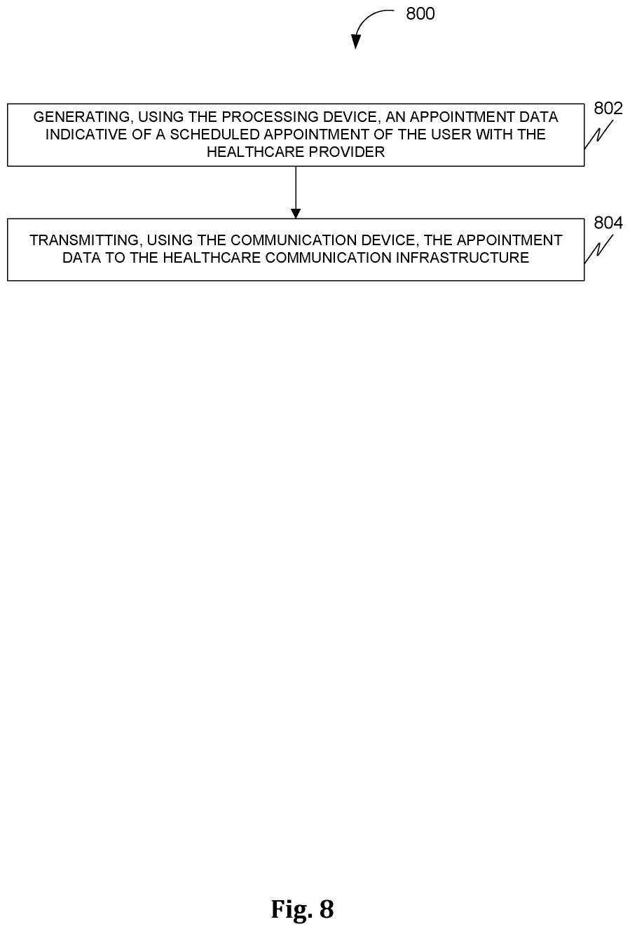

FIG. 8 illustrates a flowchart of a method 800 of facilitating a health assessment including generating, using the processing device 904, an appointment data indicative of a scheduled appointment of the user with the healthcare provider, in accordance with some embodiments.

Further, in some embodiments, the method 800 may include a step 802 of generating, using the processing device 904, an appointment data indicative of a scheduled appointment of the user with the healthcare provider.

In some embodiments, the appointment data corresponds to a scheduled telephonic appointment of the user with the healthcare provider.

Further, in some embodiments, the method 800 further may include a step 804 of transmitting, using the communication device 902, the appointment data to the healthcare communication infrastructure 906.

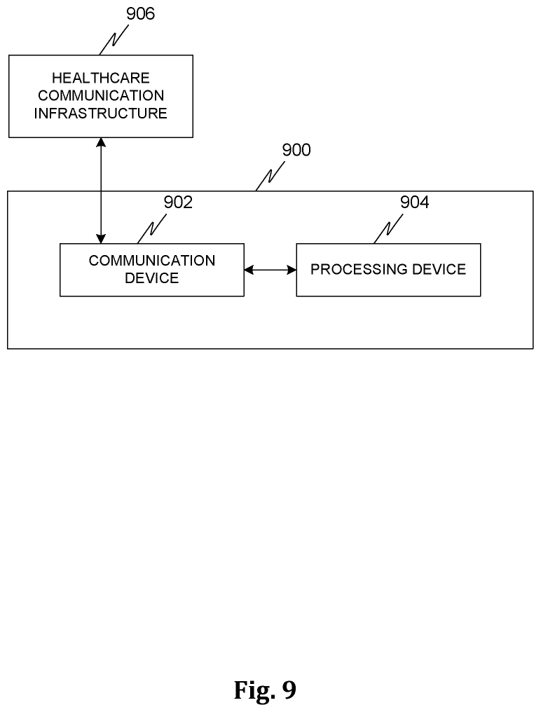

FIG. 9 illustrates a block diagram of a system 900 of facilitating a health assessment, in accordance with some embodiments.

Accordingly, the system 900 may include a communication device 902. Further, the communication device 902 may be configured to receive a medical inquiry from a healthcare communication infrastructure 906 associated with a healthcare provider. Further, the medical inquiry may be generated by a user. Further, the communication device 902 may be configured to transmit an assessment query to the healthcare communication infrastructure 906. Further, the communication device 902 may be configured to receive a response from the healthcare communication infrastructure 906. Further, the response corresponds to the assessment query. Further, the communication device 902 may be configured to transmit a diagnosis data to the healthcare communication infrastructure 906. Further, the system 900 may include a processing device 904 communicatively coupled with the communication device 902. Further, the processing device 904 may be configured to analyze the medical inquiry. Further, the processing device 904 may be configured to generate the assessment query based on the analyzing. Further, the generation of the assessment query may be based on one or more of a template and a first machine learning model. Further, the template includes a standardized assessment query. Further, the processing device 904 may be configured to analyze the response. Further, the processing device 904 may be configured to determine the diagnosis data based on the analyzing.

In some embodiments, the processing device 904 may be further configured to determine a healthcare domain associated with the medical inquiry based on the analyzing. Further, the assessment query includes two or more assessment queries associated with the healthcare domain. Further, the response includes two or more responses. Further, each of the two or more responses corresponds to each of the two or more assessment questions.

Further, in some embodiments, the processing device 904 may be further configured to generate a search query based on the analyzing of the medical inquiry. Further, the communication device 902 may be further configured to transmit the search query to an external data source device. Further, the communication device 902 may be further configured to receive an external data from the external data source device. Further, the receiving of the external data may be in response to the search query. Further, the generation of one or more of the diagnosis data and the assessment query may be further based on the external data.

In some embodiments, the first machine learning model includes a large language model. Further, the medical inquiry includes a symptom of a medical condition of the user. Further, the large language model may be configured to receive the symptom as an input. Further, the large language model may be configured to generate the assessment query as output based on the input.

Further, in some embodiments, the communication device 902 may be further configured to transmit the diagnosis data to the healthcare provider device. Further, the communication device 902 may be further configured to receive a treatment data from the healthcare provider device. Further, the treatment data may be generated by the healthcare provider based on the diagnosis data. Further, the treatment data corresponds to a treatment associated with the diagnosis data. Further, the communication device 902 may be further configured to transmit the treatment data to the healthcare communication infrastructure 906.

In some embodiments, the analyzing of the medical inquiry may be based on a speech recognition model. Further, the speech recognition model may be configured to determine one or more of a medical malady or a symptom of medical malady. Further, the generation of the assessment query may be based on one or more of the medical malady and the symptom of medical malady.

Further, in some embodiments, the processing device 904 may be further configured to determine a healthcare domain based on the analyzing of the response. Further, the processing device 904 may be further configured to generate two or more assessment queries based on the healthcare domain. Further, the processing device 904 may be further configured to analyze two or more assessment responses. Further, the generation of the diagnosis data may be further based on the analyzing of the two or more responses. Further, the communication device 902 may be further configured to. Further, the processing device 904 may be further configured to transmit the two or more assessment queries to the healthcare communication infrastructure 906. Further, the processing device 904 may be further configured to receive the two or more responses in response to the transmitting of the two or more assessment queries.

In some embodiments, the analyzing of the responses may be further based on a second machine learning. Further, the second machine learning model may be configured to receive an input based on the response. Further, the second machine learning model may be configured to generate the diagnosis data as an output.

In some embodiments, the processing device 904 may be further configured to generate an appointment data indicative of a scheduled appointment of the user with the healthcare provider. Further, the communication device 902 may be further configured to transmit the appointment data to the healthcare communication infrastructure 906.

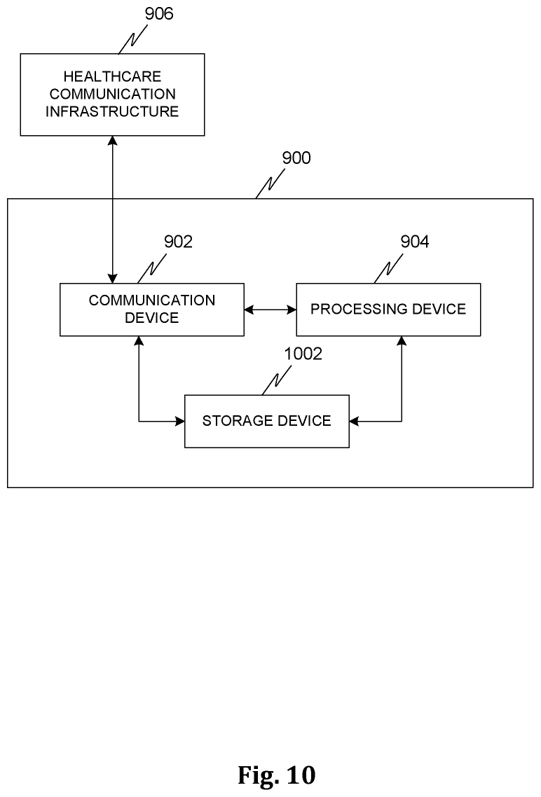

FIG. 10 illustrates a block diagram of the system 900 of facilitating a health assessment, in accordance with some embodiments.

In some embodiments, the processing device 904 may be further configured to generate a relevant healthcare provider query based on the diagnosis data. Further, the system 900 further includes a storage device 1002 which may be configured to retrieve a relevant healthcare provider data from two or more healthcare provider data stored in the storage device 1002 based on an execution of the relevant healthcare provider query. Further, the communication device 902 may be further configured to transmit the relevant healthcare provider data to the healthcare communication infrastructure 906.

Although the invention has been explained in relation to its preferred embodiment, it is to be understood that many other possible modifications and variations can be made without departing from the spirit and scope of the invention as hereinafter claimed.

1. A method of facilitating a health assessment, the method comprising:
receiving, using a communication device, a medical inquiry from a healthcare communication infrastructure associated with a healthcare provider, wherein the medical inquiry is generated by a user;
analyzing, using a processing device, the medical inquiry;
generating, using the processing device, an assessment query based on the analyzing, wherein the generation of the assessment query is based on one or more of a template and a first machine learning model, wherein the template comprises a standardized assessment query;
transmitting, using the communication device, the assessment query to the healthcare communication infrastructure;
receiving, using the communication device, a response from the healthcare communication infrastructure, wherein the response corresponds to the assessment query;
analyzing, using the processing device, the response;
determining, using the processing device, a diagnosis data based on the analyzing; and
transmitting, using the processing device, the diagnosis data to the healthcare communication infrastructure.

2. The method of claim 1 further comprises determining, using the processing device, a healthcare domain associated with the medical inquiry based on the analyzing, wherein the assessment query comprises a plurality of assessment queries associated with the healthcare domain, wherein the response comprises a plurality of responses, wherein each of the plurality of responses corresponds to each of the plurality of assessment questions.

3. The method of claim 1 further comprises:
generating, using the processing device, a search query based on the analyzing of the medical inquiry;
transmitting, using the communication device, the search query to an external data source device; and
receiving, using the communication device, an external data from the external data source device, wherein the receiving of the external data is in response to the search query, wherein the generation of one or more of the diagnosis data and the assessment query is further based on the external data.

4. The method of claim 1, wherein the first machine learning model comprises a large language model, wherein the medical inquiry comprises a symptom of a medical condition of the user, wherein the large language model is configured to receive the symptom as an input, wherein the large language model is configured to generate the assessment query as output based on the input.

5. The method of claim 1 further comprises:
transmitting, using the communication device, the diagnosis data to the healthcare provider device;
receiving, using the communication device, a treatment data from the healthcare provider device, wherein the treatment data is generated by the healthcare provider based on the diagnosis data, wherein the treatment data corresponds to a treatment associated with the diagnosis data; and
transmitting, using the communication device, the treatment data to the healthcare communication infrastructure.

6. The method of claim 1 further comprises:
generating, using the processing device, a relevant healthcare provider query based on the diagnosis data;
retrieving, using a storage device, a relevant healthcare provider data from a plurality of healthcare provider data based on execution of the relevant healthcare provider query; and
transmitting, using the communication device, the relevant healthcare provider data to the healthcare communication infrastructure.

7. The method of claim 1, wherein the analyzing of the medical inquiry is based on a speech recognition model, wherein the speech recognition model is configured to determine one or more of a medical malady or a symptom of medical malady, wherein the generation of the assessment query is based on one or more of the medical malady and the symptom of medical malady.

8. The method of claim 1 further comprises:
determining, using the processing device, a healthcare domain based on the analyzing of the response;
generating, using the processing device, a plurality of assessment queries based on the healthcare domain;
transmitting, using the communication device, the plurality of assessment queries to the healthcare communication infrastructure;
receiving, using the communication device, a plurality of responses in response to the transmitting of the plurality of assessment queries;
analyzing, using the processing device, the plurality of assessment queries, wherein the generation of the diagnosis data is further based on the analyzing of the plurality of responses.

9. The method of claim 1, wherein the analyzing of the responses is further based on a second machine learning, wherein the second machine learning model is configured to receive an input based on the response, wherein the second machine learning model is configured to generate the diagnosis data as an output.

10. The method of claim 1 further comprises:
generating, using the processing device, an appointment data indicative of a scheduled appointment of the user with the healthcare provider;
transmitting, using the communication device, the appointment data to the healthcare communication infrastructure.

11. A system for facilitating a health assessment, the system comprising:
a communication device configured to:
receive a medical inquiry from a healthcare communication infrastructure associated with a healthcare provider, wherein the medical inquiry is generated by a user;
transmit an assessment query to the healthcare communication infrastructure;
receive a response from the healthcare communication infrastructure, wherein the response corresponds to the assessment query;
transmit a diagnosis data to the healthcare communication infrastructure;
a processing device communicatively coupled with the communication device, wherein the processing device is configured to:
analyze the medical inquiry;
generate the assessment query based on the analyzing, wherein the generation of the assessment query is based on one or more of a template and a first machine learning model, wherein the template comprises a standardized assessment query;
analyze the response; and
determine the diagnosis data based on the analyzing.

12. The system of claim 11, wherein the processing device is further configured to determine a healthcare domain associated with the medical inquiry based on the analyzing, wherein the assessment query comprises a plurality of assessment queries associated with the healthcare domain, wherein the response comprises a plurality of responses, wherein each of the plurality of responses corresponds to each of the plurality of assessment questions.

13. The system of claim 11, wherein the processing device is further configured to generate a search query based on the analyzing of the medical inquiry, wherein the communication device is further configured to:
transmit the search query to an external data source device; and
receive an external data from the external data source device, wherein the receiving of the external data is in response to the search query, wherein the generation of one or more of the diagnosis data and the assessment query is further based on the external data.

14. The system of claim 11, wherein the first machine learning model comprises a large language model, wherein the medical inquiry comprises a symptom of a medical condition of the user, wherein the large language model is configured to receive the symptom as an input, wherein the large language model is configured to generate the assessment query as output based on the input.

15. The system of claim 11, wherein the communication device is further configured to:
transmit the diagnosis data to the healthcare provider device;
receive a treatment data from the healthcare provider device, wherein the treatment data is generated by the healthcare provider based on the diagnosis data, wherein the treatment data corresponds to a treatment associated with the diagnosis data; and
transmit the treatment data to the healthcare communication infrastructure.

16. The system of claim 11, wherein the processing device is further configured to generate a relevant healthcare provider query based on the diagnosis data, wherein the system further comprises a storage device configured to retrieve a relevant healthcare provider data from a plurality of healthcare provider data stored in the storage device based on execution of the relevant healthcare provider query, wherein the communication device is further configured to transmit the relevant healthcare provider data to the healthcare communication infrastructure.

17. The system of claim 11, wherein the analyzing of the medical inquiry is based on a speech recognition model, wherein the speech recognition model is configured to determine one or more of a medical malady or a symptom of medical malady, wherein the generation of the assessment query is based on one or more of the medical malady and the symptom of medical malady.

18. The system of claim 11, wherein the processing device is further configured to:
determine a healthcare domain based on the analyzing of the response;
generate a plurality of assessment queries based on the healthcare domain;
analyze a plurality of assessment responses, wherein the generation of the diagnosis data is further based on the analyzing of the plurality of responses, wherein the communication device is further configured to:
transmit the plurality of assessment queries to the healthcare communication infrastructure;
receive the plurality of responses in response to the transmitting of the plurality of assessment queries.

19. The system of claim 11, wherein the analyzing of the responses is further based on a second machine learning, wherein the second machine learning model is configured to receive an input based on the response, wherein the second machine learning model is configured to generate the diagnosis data as an output.

20. The system of claim 11, wherein the processing device is further configured to generate an appointment data indicative of a scheduled appointment of the user with the healthcare provider, wherein the communication device is further configured to transmit the appointment data to the healthcare communication infrastructure.
